(19) United States
(12) Patent Application Publication
(10) Pub. No.: US 2022/0118681 A1
(43) Pub. Date: Apr. 21, 2022

(54) Printhead Assembly for a 3D Bioprinter

(71) Applicant: Inventia Life Science Pty Ltd, Alexandria, New South Wales (AU)

(72) Inventors: Andrew Sexton, Coogee, New South Wales (AU); Aidan O'Mahony, Coogee, New South Wales (AU); Zachary Artist, Neutral Bay, New South Wales (AU); William Lim, Gordon, New South Wales (AU); Samuel Myers, Blackheath, New South Wales (AU)

(73) Assignee: Inventia Life Science Pty Ltd, Alexandria, New South Wales (AU)

(21) Appl. No.: 17/298,166

(22) PCT Filed: Dec. 6, 2019
(86) PCT No.: PCT/AU2019/051336

§ 371 Date: May 28, 2021

(30) Foreign Application Priority Data
Dec. 6, 2018 (AU) 2018904641

(51) Int. Cl. B29C 64/112; B29C 64/209; B29C 64/307; C12N 5/071

(52) U.S. Cl. CPC B29C 64/112; B29C 64/209; B33Y 10/00; C12N 5/0602; B29C 64/307

(57) ABSTRACT

A printhead assembly (100) suitable for a 3D bioprinter is disclosed, the printhead assembly (100) comprising at least one reservoir (106); a sample loading system (102) in fluid communication with the at least one reservoir (106), the sample loading system (102) configured to direct fluid into the at least one reservoir (106); and a dispensing system (103) having at least one dispensing outlet (126), the at least one dispensing outlet (126) in fluid communication with the at least one reservoir (106) and configured to dispense fluid from the at least one reservoir (106).

### CROSS REFERENCE TO RELATED APPLICATION

This application claims priority to Australian Provisional Patent Application No 2018904641 filed 6 Dec. 2018, the contents of which are incorporated herein by reference in its entirety.

### TECHNICAL FIELD

The technology relates to a printhead assembly for 3D printers suitable for printing cells and reagents.

### BACKGROUND

The workhorse of in vitro cell biology is cell culture where primary or immortalized cells are simply plated onto plastic or glass surfaces. A number of cellular properties, such as in cell proliferation, differentiation and responses towards external stimuli, are fundamentally different for cells in 2D and the 3D environments found in vivo. Particularly for drug development and precision medicine programs, cell culture conditions that better reflect the 3D animal environments, and hence would limit the number of failed animal experiments, would be highly advantageous.

For example, in cancer cell biology, 3D models exhibit more in vivo tumor-like features including hypoxic regions, gradient distribution of chemical and biological factors and expression of pro-angiogenic and multidrug resistance proteins, compared to 2D cell culture models.

It is for this reason that 3D multicellular models, are generally regarded as superior models of in vivo systems than the more popular 2D cell culture. Further, most cellular structures in multicellular biology are organised three-dimensionally.

There exist many commercially available 3D bioprinters, for example: 3D-Bioplotter® by EnvisionTEC; BioScaffolder by GeSiM; Bio X by Cellink; BioFactory® by RegenHU; BioBot 2 by BioBots. The commercially available 3D bioprinters are most commonly based on micro-extrusion, thermal inkjet or piezoelectric inkjet technology. The commercially available 3D bioprinters most commonly utilise cartridges (e.g. Nordson Optimum® Syringe Barrels) for loading substances into the printer. Each one of these cartridges is often coupled to a single printhead. Maintenance of sterility is challenging during cartridge filling, handling, installation and removal.

The design of 3D models of organ or tissue architecture for 3D bioprinting applications have largely been based on:

a) noninvasive medical imaging technologies (e.g. computed tomography (CT) and magnetic resonance imaging (MRI)) for data collection; and

b) computer-aided design and computer-aided manufacturing (CAD-CAM) tools and mathematical modelling for information digitisation, generation of 3D-rendered models and generation of 2D cross-sectional images.

There is a need for tools and techniques that facilitate application of 3D cell culture models in a scalable, repeatable and cost-effective manner to drug discovery, personalized medicine and general cell biology.

The present inventors have developed printhead assembly for 3D bioprinters suitable for printing cells and reagents.

### SUMMARY

In a first aspect, there is provided a printhead assembly suitable for a 3D bioprinter, the printhead assembly comprising:

a reservoir;

a sample loading system in fluid communication with the reservoir, the sample loading system configured to direct fluid into the reservoir; and

a dispensing system having a dispensing outlet, the dispensing outlet in fluid communication with the reservoir and configured to dispense fluid from the reservoir.

In an embodiment,

the reservoir is one of a plurality of reservoirs;

the sample loading system is in fluid communication with each reservoir and is configured to direct fluid into any one of the plurality of reservoirs;

the dispensing outlet is one of a plurality of dispensing outlets; and

each dispensing outlet is in fluid communication with one of the plurality of reservoirs and is configured to dispense fluid from the respective reservoir.

In an embodiment, the sample loading system is configured to draw a fluid from a container and prime any one of the plurality of reservoirs with the fluid.

In an embodiment, the sample loading system comprises a manifold in fluid communication with the plurality of reservoirs, the manifold configured to direct fluid into any one of the plurality of reservoirs.

In an embodiment, the sample loading system further comprises a plurality of priming fluid lines, each priming fluid line coupling one reservoir in fluid communication to the manifold.

In an embodiment:

each reservoir has a reservoir outlet and a reservoir inlet;

each dispensing outlet is in fluid communication with the reservoir outlet of one of the plurality of reservoirs; and

each priming fluid line is in fluid communication with the manifold and the reservoir inlet of one of the plurality of reservoirs.

In an embodiment, each dispensing outlet is coupled in fluid communication to the reservoir outlet of one of the plurality of reservoirs by a dispensing fluid line.

In an embodiment, each dispensing fluid line comprises a particulate trap configured to reduce particulates from settling in the respective dispensing outlet.

In an embodiment, the particulate trap is formed by one or more loops in the dispensing line.

In an embodiment, each priming fluid line comprises a valve having:

an open configuration that allows fluid to flow from the manifold into the respective reservoir; and

a closed configuration that prevents fluid flowing from the manifold into the respective reservoir.

In an embodiment, the sample loading system comprises a pump coupled in fluid communication to an inlet of the manifold, the pump configured to draw fluid into the sample loading system and pump the fluid out of the sample loading system into any one of the reservoirs.

In an embodiment, the sample loading system comprises a manifold valve in fluid communication with an inlet of the manifold, the manifold valve having:

an open configuration that allows fluid to flow into the manifold through the inlet of the manifold; and

a closed configuration that prevents fluid flowing into the manifold through the inlet of the manifold.

In an embodiment, the manifold valve in the closed configuration prevents fluid flowing out of the manifold through the inlet of the manifold.

In an embodiment, the sample loading system further comprises a needle in fluid communication with the inlet of the manifold, the needle configured to be inserted into a container to draw fluid from the container.

In an embodiment, the sample loading system further comprises an actuator configured to insert the needle into a container to draw fluid from the container and to withdraw the needle from the container.

In an embodiment, the manifold has a sensor configured to detect fluid flowing out of an outlet of the manifold.

In an embodiment, each reservoir is configured to be coupled in fluid communication to a pressurized source of gas to pressurize each reservoir.

In an embodiment, each reservoir is configured to be coupled to a pressure regulator to regulate the pressure in the respective reservoir.

In an embodiment, each dispensing outlet is a nozzle having:

an open configuration that allows fluid to be dispensed from the respective reservoir; and

a closed configuration that prevents fluid from being dispensed from the respective reservoir.

In an embodiment, the printhead assembly further comprises a housing in which each reservoir, the sample loading system, and the dispensing system are disposed.

In an embodiment, the sample loading system is configured to be coupled in fluid communication to a pump, the pump being configured to draw fluid into the sample loading system and pump the fluid out of the sample loading system into any one of the reservoirs.

In an embodiment, the printhead assembly further comprises an electronics assembly configured to control operation of the printhead assembly.

In a second aspect, there is provided a 3D bioprinter for printing cells, the bioprinter comprising:

a printhead assembly according to the first aspect;

a print stage for locating a substrate on which a 3D cell construct can be fabricated; and

a cartridge receptacle.

There is disclosed a 3D bioprinter for printing cells, the bioprinter comprising:

a printhead assembly according to the first aspect;

a print stage for locating a substrate on which a 3D cell construct can be fabricated;

a cartridge receptacle; and

a pump in fluid communication with the sample loading system, the pump configured to draw fluid into the sample loading system and pump the fluid out of the sample loading system into any one of the reservoirs.

In an embodiment, the bioprinter further comprises a housing in which the printhead assembly, the print stage, and the cartridge receptacle are disposed.

In an embodiment, the housing has an access door having an open position that permits access to an interior of the bioprinter and a closed position that prevents access to the interior of the bioprinter.

In an embodiment, the bioprinter further comprises a pressure regulating system coupled in fluid communication to each reservoir to regulate the pressure in each reservoir, and the pressure regulating system configured to be coupled in fluid communication to a source of pressurized gas for pressurizing each reservoir.

In an embodiment, the pressure regulating system comprises a connector configured to couple the pressure regulating system in fluid communication to a source of pressurized gas.

In an embodiment, the connector projects from the housing.

In an embodiment, the pressure regulating system comprises a regulator manifold in fluid communication with each reservoir, the regulator manifold configured to be coupled in fluid communication to a source of pressurized gas.

In an embodiment, each reservoir is coupled in fluid communication to the regulator manifold by a pressure regulator, each pressure regulator configured to regulate the pressure in the respective reservoir.

In an embodiment, the further comprises a selector valve coupling the pump in fluid communication to the sample loading system and coupling each reservoir in fluid communication to the pressure regulating system and the pump.

In an embodiment, the bioprinter further comprises an air flow system disposed in the housing, the air flow system configured to induce an air flow within the housing.

In an embodiment, the air flow system is configured to draw air underneath the print stage and the cartridge receptacle.

In an embodiment, the air flow system comprises a blower to induce the air flow within the housing.

In an embodiment, the air flow system comprises at least one high efficiency particulate arresting filter.

In an embodiment, the bioprinter further comprises a holder in which the cartridge receptacle and the print stage are located.

In an embodiment, the bioprinter further comprises a first positioning unit having a track, the first positioning unit coupled to the holder and configured to position the holder along the track of the first positioning unit.

In an embodiment, the bioprinter further comprises a second positioning unit having a track, the second positioning unit coupled to the printhead assembly and configured to position the printhead assembly along the track of the second positioning unit.

In an embodiment, the track of the first positioning unit extends at least substantially perpendicular to the track of the second positioning unit.

In an embodiment, the bioprinter further comprises a control system to control operation of the bioprinter.

In an embodiment, the control system comprises a reader, and the control system is configured to use the reader to read an identifier of a cartridge inserted into the cartridge receptacle to obtain information about the cartridge.

In an embodiment, the information about the cartridge includes information about what fluids are contained in the cartridge, in which container particular fluids are located, whether the cartridge has been used, and/or whether the cartridge is unused.

In an embodiment, the reader is a Radio-Frequency Identification (RFID) reader and the identifier is an RFID tag or label.

In an embodiment, the reader is a read/write RFID reader, the identifier is a rewritable RFID tag or label, and the control system is configured to use the read/write RFID reader to obtain information from the rewritable RFID tag or label and write/rewrite information on the rewritable RFID tag or label.

In an embodiment, the control system comprises a user interface, the user interface configured to permit a user to input information and control instructions into the control system for a particular print job.

In a third aspect, there is provided a method of printing a three-dimensional (3D) cell construct by dispensing a plurality of fluid droplets from the dispensing system of a printhead according to the first aspect.

In a fourth aspect there is provided a method of fabricating a three-dimensional (3D) cell construct by dispensing a plurality of fluid droplets from the dispensing system of a bioprinter according to the second aspect.

An advantage of the present technology is that it allows printing of cells without causing issues with cell viability and activity after printing or forming 3D cell structures.

### Definitions

Throughout this specification, unless the context clearly requires otherwise, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Throughout this specification, the term ‘consisting of’ means consisting only of.

Any discussion of documents, acts, materials, devices, articles or the like which has been included in the present specification is solely for the purpose of providing a context for the present technology. It is not to be taken as an admission that any or all of these matters form part of the prior art base or were common general knowledge in the field relevant to the present technology as it existed before the priority date of each claim of this specification.

Unless the context requires otherwise or specifically stated to the contrary, integers, steps, or elements of the technology recited herein as singular integers, steps or elements clearly encompass both singular and plural forms of the recited integers, steps or elements.

In the context of the present specification the terms ‘a’ and ‘an’ are used to refer to one or more than one (ie, at least one) of the grammatical object of the article. By way of example, reference to ‘an element’ means one element, or more than one element.

In the context of the present specification the term ‘about’ means that reference to a figure or value is not to be taken as an absolute figure or value, but includes margins of variation above or below the figure or value in line with what a skilled person would understand according to the art, including within typical margins of error or instrument limitation. In other words, use of the term ‘about’ is understood to refer to a range or approximation that a person or skilled in the art would consider to be equivalent to a recited value in the context of achieving the same function or result.

Those skilled in the art will appreciate that the technology described herein is susceptible to variations and modifications other than those specifically described. It is to be understood that the technology includes all such variations and modifications. For the avoidance of doubt, the technology also includes all of the steps, features, and compounds referred to or indicated in this specification, individually or collectively, and any and all combinations of any two or more of said steps, features and compounds.

### BRIEF DESCRIPTION OF THE DRAWINGS

Preferred embodiments of the present invention will now be described, by way of examples only, with reference to the accompanying drawings, in which:

FIG. 1 is an isometric view of a printhead assembly according to a first embodiment of the present invention, a cartridge, a substrate, and a holder of a bioprinter that is capable of being used with the printhead assembly;

FIG. 2 is an isometric view of the printhead assembly of FIG. 1 having an access panel removed;

FIG. 3 is an isometric view of the printhead assembly of FIG. 1, omitting the housing of the printhead assembly;

FIG. 4 is a front view of the printhead assembly of FIG. 1 having the access panel removed;

FIG. 5 is a bottom view of the printhead assembly of FIG. 1;

FIG. 6 is a front isometric view of a bioprinter including the printhead assembly of FIG. 1;

FIG. 7 is a rear isometric view of the bioprinter of FIG. 6;

FIG. 8 is a front isometric view of the bioprinter of FIG. 6, wherein the housing of the bioprinter and the housing of the printhead assembly are illustrated with an outline only;

FIG. 9 is an exploded parts view of the cartridge of FIG. 1;

FIG. 10 is a top view of the cartridge, the substrate, and the holder of FIG. 1;

FIG. 11 is a front isometric view illustrating the printhead assembly of FIG. 1 and the positioning units, the pressure regulating system, and the selector valve of the bioprinter of FIG. 6;

FIG. 12 is a rear isometric view of the bioprinter of FIG. 6, wherein the housing of the bioprinter is illustrated with an outline only;

FIG. 13 is a rear isometric view illustrating the printhead assembly of FIG. 1 and the positioning units, the pressure regulating system, and the selector valve of the bioprinter of FIG. 6;

FIG. 14 is a rear isometric view of the pump, the selector valve, the printhead without the printhead body, and the cartridge of the bioprinter of FIG. 6;

FIG. 15 is a front isometric view of the pump, the selector valve, the printhead without the printhead body, and the cartridge of the bioprinter of FIG. 6;

FIG. 16 is a rear isometric view of the laminar air flow system of the bioprinter of FIG. 6;

FIG. 17 is another rear isometric view of the laminar air flow system of the bioprinter of FIG. 6;

FIG. 18 is a schematic of the air flow through the laminar air flow system of FIGS. 16 and 17;

FIG. 19 is a schematic of the bioprinter of FIG. 6;

FIG. 20 is a screenshot of the Graphical User Interface (GUI) of the bioprinter of FIG. 6;

FIG. 21 is another screenshot of the GUI of the bioprinter of FIG. 6;

FIG. 22 is a flow chart for fabricating a three-dimensional cell construct using the bioprinter of FIG. 6;

FIG. 23 is a front view of a printhead assembly according to a second embodiment of the present invention;

FIG. 24 is a bottom view of the printhead assembly of FIG. 23;

FIG. 25 is an isometric view of the printhead assembly of FIG. 23, omitting the housing of the printhead assembly;

FIG. 26 is a schematic of an alternative embodiment of the printhead assembly of FIG. 23;

FIG. 27 is a schematic of another alternative embodiment of the printhead assembly of FIG. 23

FIGS. 28A-E illustrate the problem of cells settling in dead zones of the dispensing outlets of the printhead assemblies of FIGS. 1 and 23;

FIGS. 29A-E illustrate an example dispensing line according to an embodiment that reduces cells settling in the dead zones of the dispensing outlets of the printhead assemblies of FIGS. 1 and 23;

FIGS. 30A-C show example dispensing lines according to another embodiment that reduce cells settling in the dead zones of the dispensing outlets of the printhead assemblies of FIGS. 1 and 23; and

FIG. 31 shows an example dispensing line according to another embodiment that reduces cells settling in the dispensing outlets of the printhead assemblies of FIGS. 1 and 23.

### DETAILED DESCRIPTION OF EMBODIMENTS

### First Exemplary Embodiment of the Printhead Assembly

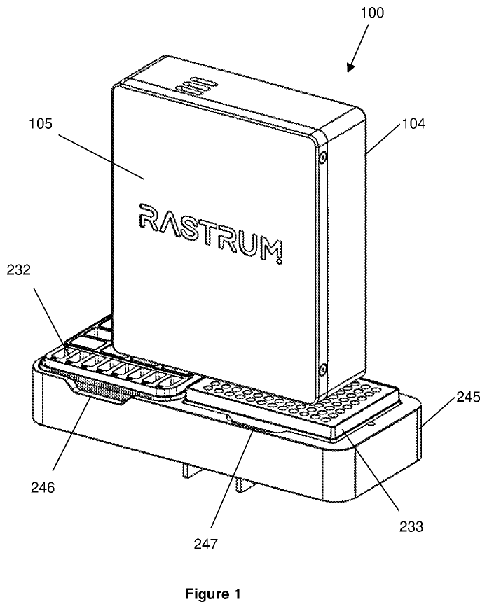

FIGS. 1 to 5 show a printhead assembly 100 according to a first embodiment of the present invention. The printhead assembly 100 has a first and a second set of reservoirs 101, a sample loading system 102, and a dispensing system 103, all of which are disposed in a printhead housing 104. Removing an access panel 105 of the printhead housing 104 permits access to the first and the second set of reservoirs 101, the sample loading system 102, and the dispensing system 103. Both the first and the second sets of reservoirs 101 have four reservoirs 106, however, each set of reservoirs 101 may have more or less than four reservoirs 106.

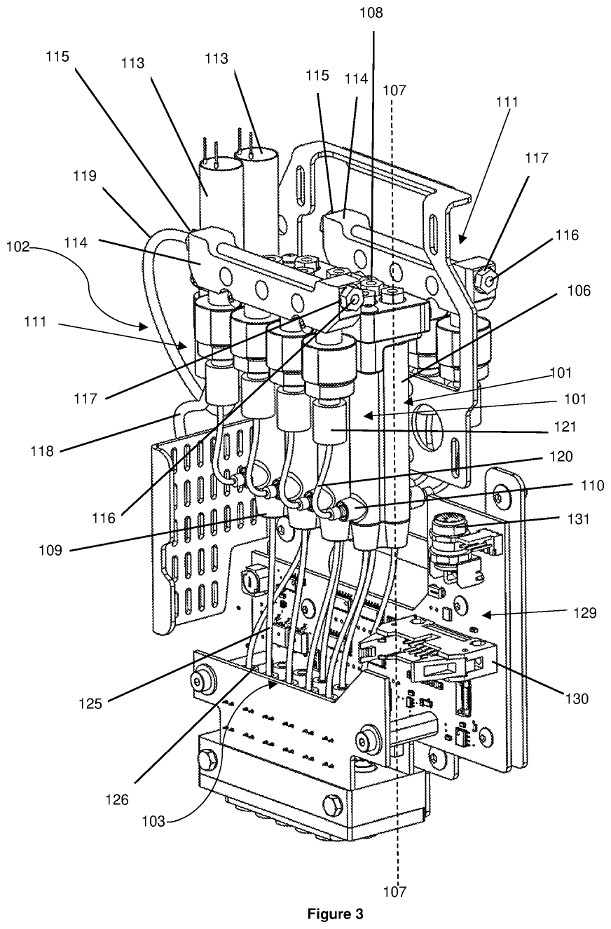

Referring to FIG. 3, each reservoir 106 has a longitudinal axis 107 extending substantially vertically, a cap 108 located at the top of the reservoir 106, a reservoir outlet 109 located at a lower region of the reservoir 106, and a reservoir inlet 110 located at a predetermined height above the reservoir outlet 109. For each reservoir 106, the cap 108, the reservoir outlet 109, and the reservoir inlet 110 are in fluid communication with the interior of the reservoir 106.

Referring to FIGS. 3 to 5, the sample loading system 102 has a first and a second subsystem 111. Each subsystem 111 is in fluid communication with either the first or the second set of reservoirs 101. Each subsystem 111 of the sample loading system 102 comprises a needle 112, a manifold valve 113, and a priming manifold 114. Each priming manifold 114 has a manifold inlet 115 and a manifold outlet 116. For each subsystem 111, the needle 112 is coupled in fluid communication to the manifold valve 113 by a fluid line 118 and the manifold valve 113 is coupled in fluid communication to the manifold inlet 115 of the priming manifold 114 by a fluid line 119. Accordingly, for each subsystem 111, the needle 112, the manifold valve 113, and the priming manifold 114 are all in fluid communication with each other.

The manifold valve 113 of each subsystem 111 has an open configuration and a closed configuration. In the open configuration, the manifold valve 113 of each subsystem 111 allows fluid to flow from the needle 112 into the priming manifold 114 through the manifold inlet 115. In the closed configuration, the manifold valve 113 of each subsystem 111 prevents fluid flowing from the needle 112 to the manifold inlet 115 and prevents fluid flowing out of the priming manifold 114 through the manifold inlet 115 towards the needle 112. It is envisaged that the manifold valves 113 may be normally closed solenoid valves, however, it will be appreciated that any other suitable valves/nozzles known in the art may be used.

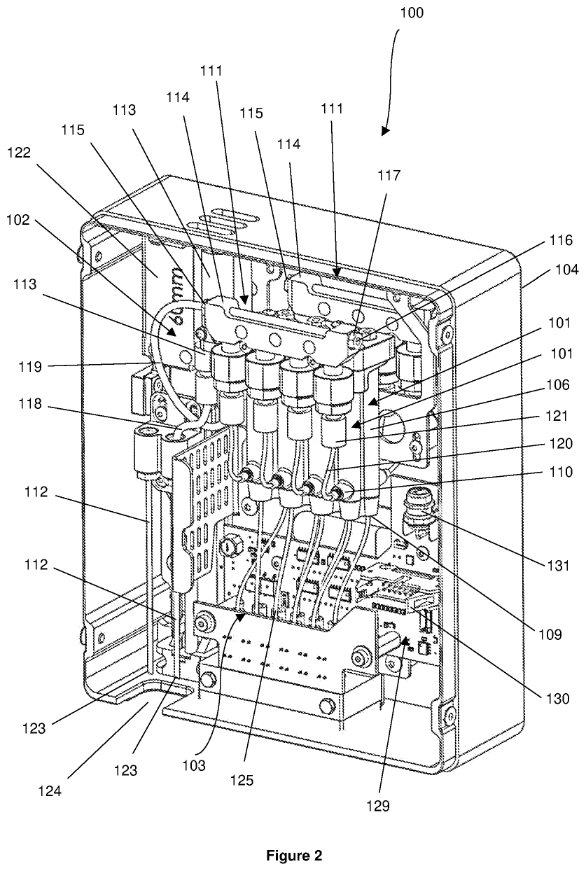

Referring to FIGS. 2 to 4, a sensor 117 is disposed at the manifold outlet 116 of each priming manifold 114. For each priming manifold 114, the sensor 117 is configured to detect fluid flowing out of the priming manifold 114 through the manifold outlet 116. Alternatively, for each priming manifold 114, a sensor 117 may be disposed at the manifold inlet 115 and configured to detect fluid flowing into the priming manifold 114 through the manifold inlet 115. For each priming manifold 114, it is also envisaged that a sensor 117 may be disposed at the manifold inlet 115 that is configured to detect fluid flowing into the priming manifold 114 through the manifold inlet 115 and that a sensor 117 may be disposed at the manifold outlet 116 that is configured to detect fluid flowing out of the manifold 114 through the manifold outlet 116. The sensors 117 may be optical sensors, however, any other suitable sensors known in the art that may be used.

The reservoir inlet 110 of each reservoir 106 is coupled in fluid communication to one of the priming manifolds 114 by a priming fluid line 120 having a check valve 121. For each priming fluid line 120, the check valve 121 has an open position and a closed position. In the open position, the check valve 121 permits fluid to flow from the respective priming manifold 114 through the priming fluid line 120 and into the respective reservoir 106. In the closed position, the check valve 121 prevents fluid flowing from the priming fluid line 120 into the respective priming manifold 114 and prevents fluid flowing from the respective priming manifold 114 to the respective priming fluid line 120. It is envisaged that any other suitable valves known in the art that are capable of performing the same, or similar, functions as the check valves 121 may be used. For example, active valves that can be opened and closed via a control system may be used.

It will be appreciated that each subsystem 111 of the sample loading system 102 is in fluid communication with one set of reservoirs 101 and is capable of directing fluid from the needle 112 into any one of the reservoirs 106 of the respective set of reservoir 101.

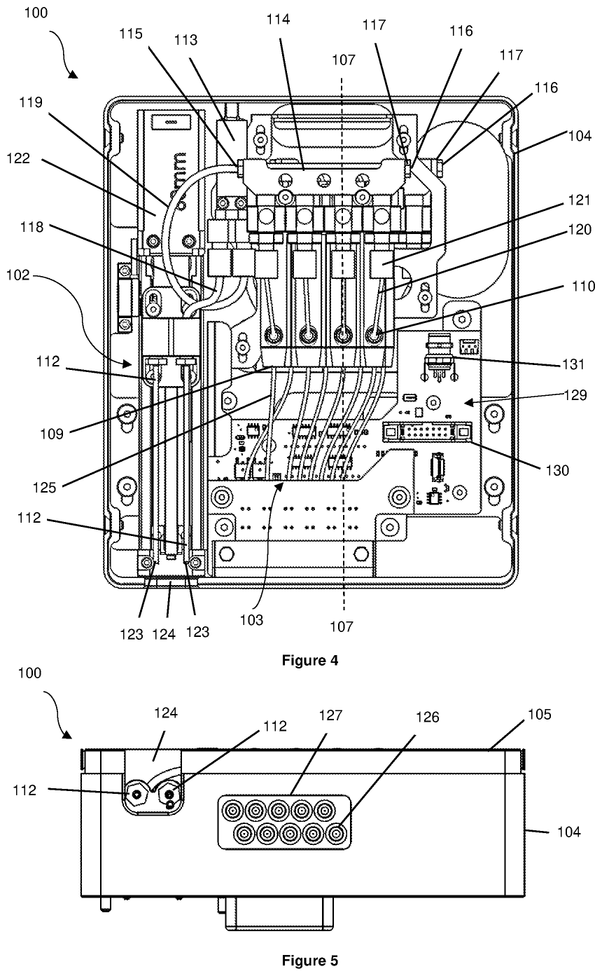

Referring to FIGS. 4 and 5, the sample loading system 102 has an actuator 122 coupled to both needles 112. The actuator 122 is configured to advance the needles 112 such that the points 123 of the needles 112 protrude from an opening 124 in the printhead housing 104. The actuator 122 is also configured to retract the needles 112 back into the printhead housing 104 through the opening 124 such that the points 123 of the needles 112 are located within the printhead housing 104. Although the actuator 122 is described and illustrated as advancing and retracting the needles 112 simultaneously, it is also envisaged that each needle 112 may have an actuator 122, such that each needle 112 may be advanced and retracted independently.

Referring to FIGS. 3 and 5, the dispensing system 103 comprises a plurality of dispensing fluid lines 125, each of which are coupled in fluid communication with the reservoir outlet 109 of one of the reservoirs 106. Coupled in fluid communication to each dispensing fluid line 125 is a dispensing outlet 126 in the form of a nozzle having a normally closed configuration and an open configuration. For each dispensing fluid line 125, when the dispensing outlet 126 is in the open configuration, fluid is allowed to flow out of the respective reservoir 106 through the reservoir outlet 109, through the dispensing fluid line 125, to be dispensed from the dispensing outlet 126. For each dispensing fluid line 125, when the dispensing outlet 126 is in the closed configuration, fluid is prevented from being dispensed from the dispensing outlet 126. It is envisaged that each dispensing outlet 126 may be a micro-solenoid valve, however, any other suitable valves known in the art may also be used.

Referring to FIG. 5, the dispensing outlets 126 are aligned with a hole 127 in the printhead housing 104 such that each dispensing outlet 126 is configured to dispense fluid out of the printhead assembly 100 through the hole 127.

Referring to FIG. 3, for each reservoir 106, the volume of the dispensing fluid line 125 and the volume between the reservoir inlet 110 and the reservoir outlet 109 within the reservoir 106 define a predetermined volume. The predetermined volume can be increased or decreased by increasing or decreasing the height difference between the reservoir outlet 109 and the reservoir inlet 110 for each reservoir 106, respectively. The predetermined volume can also be increased or decreased by increasing or decreasing the volume of the dispensing fluid line 125. It will be appreciated that increasing the predetermined volume will reduce, or possibly prevent, fluid flowing from within the reservoir 106 back up the respective priming fluid line 120.

The printhead assembly 100 further comprises an electronics assembly 129 electrically connected to each manifold valve 113, each sensor 117, each dispensing outlet 126, and the actuator 122. The electronics assembly 129 is configured to move each manifold valve 113 and each dispensing outlet 126 between their respective open and closed configurations. The electronics assembly 129 is also configured to control the actuator 122 to advance the points 123 of the needles 112 out of the printhead housing 104 and to retract the points 123 of the needles 112 back into the printhead housing 104.

The electronics assembly 129 has an electrical port 130 configured to electrically connect the electronics assembly 129 to a control system 272 (discussed below). The electronics assembly 129 also has an electrical connector 131 that is capable of being electrically connected to other electrical equipment that is internal or external to the printhead assembly 100. It is envisaged that the electronics assembly 129 may or may not include the electrical connector 131.

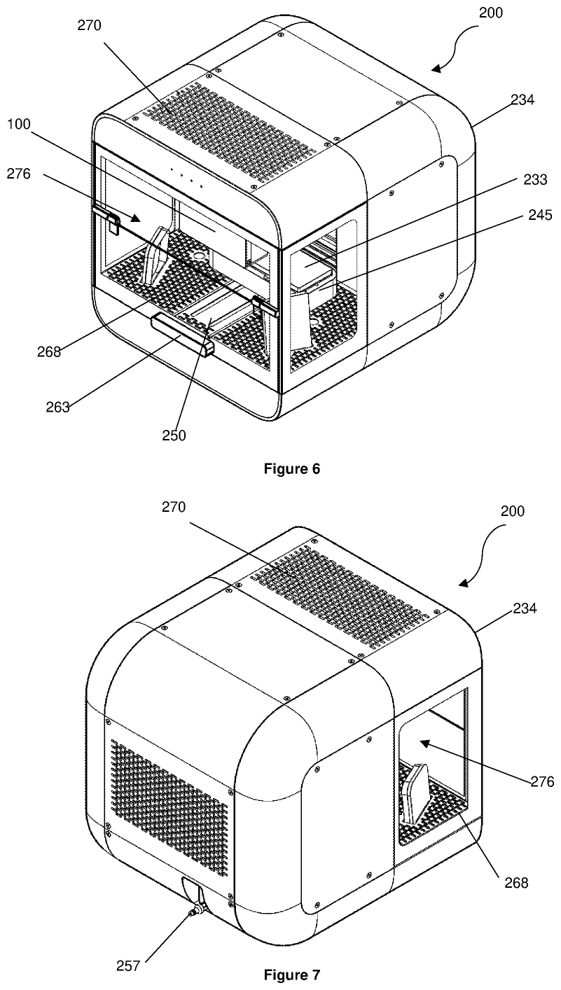

FIGS. 6 to 8 show a bioprinter 200 for fabricating three-dimensional (3D) cell constructs using the printhead assembly 100. The bioprinter 200 has a printhead assembly 100 for printing 3D cell constructs, a removable cartridge 232, and a removable substrate 233 on/in which 3D cell constructs are to be printed. The printhead assembly 100, the cartridge 232, and the substrate 233 are disposed within a housing 234.

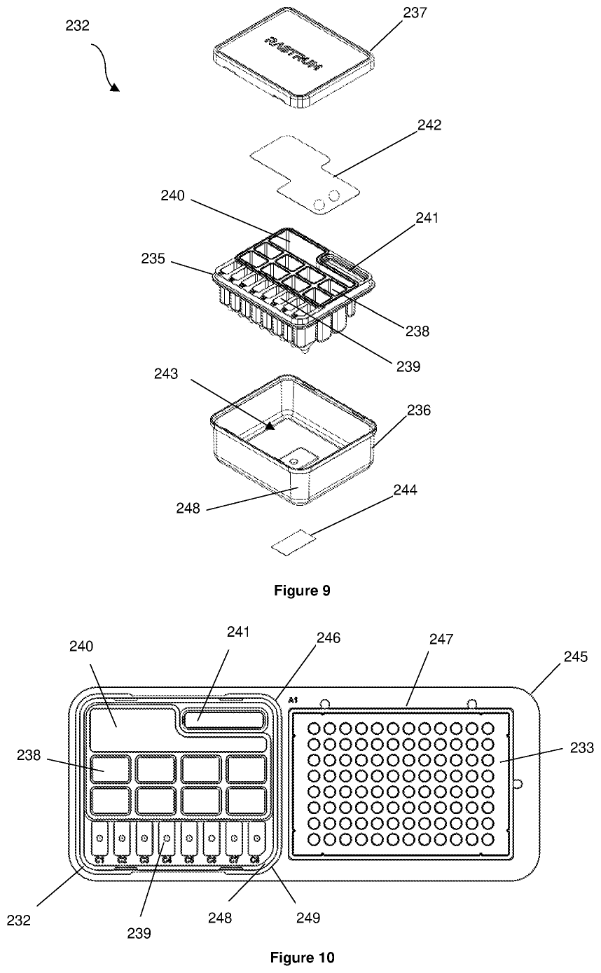

Referring to FIGS. 9 and 10, the cartridge 232 comprises a tray 235, a base 236, and a lid 237 configured to removably engage the base 236.

The tray 235 has a plurality of sealed containers 238, a plurality of unsealed containers 239, a cleaning container 240, and a waste slot 241. Each of the plurality of sealed containers 238 may contain a fluid such as, for example, a bio-ink, or an activator (both of these are described in more detail below). The plurality of unsealed containers 239 are configured to receive a fluid chosen by a user such as, for example, a cell suspension, a cell culture media, cell-ink, cell-culture solutions, or a drug in solution. The cleaning container 240 contains a cleaning fluid such as, for example, water or ethanol.

The plurality of sealed containers 238 and the cleaning container 240 are sealed by a seal 242 that is coupled to the tray 235. The seal 242 may be a film that is heat sealed onto the tray 235, however, any other suitable seals known in the art that are capable of sealing the plurality of sealed containers 238 and the cleaning container 240 may also be used.

The base 236 has an interior space 243 and an identifier 244 coupled to an external surface of the base 236. The identifier 244 may contain information about the cartridge 232 such as, for example, what fluids are contained in the cartridge 232, in which of the plurality of sealed containers 238 particular fluids are located, whether the cartridge 232 has been used, and/or whether the cartridge 232 is unused. The identifier 244 may be either a read-only Radio-Frequency Identification (RFID) or Near-Field Communication (NFC) tag or label, or a rewritable RFID or NFC tag or label.

The tray 235 is configured to be received in the interior space 243 of the base 236 and be removably coupled to the base 236. When the tray 235 is removably coupled to the base 236, the underside of the tray 235 and the interior surface of the base 236 define a waste volume (not shown) within the interior space 243 of the base 236 that is in fluid communication with the waste slot 241 of the tray 235. Accordingly, fluids passing through the waste slot 241 will be collected in the waste volume of the base 236. The base 236 is sized such that the waste volume is greater than the combined volume of the sealed containers 238, the unsealed containers 239, and the cleaning container 240. The waste volume is therefore large enough to receive the fluid contents of all the sealed containers 238, the unsealed containers 239, and the cleaning container 240.

When the tray 235 is received in the interior space 243 of the base 236 and the lid 237 is removably coupled to the base 236, the tray 235 is enclosed in a chamber defined by the base 236 and the lid 237.

Referring to FIGS. 1 and 10, the printhead assembly 100 is configured to print a 3D cell construct onto the substrate 233, which is a well-plate having 96 wells. However, multi well-plates having more or less wells may also be used. It is also envisaged that the printhead assembly 100 is configured to print a 3D cell construct onto a petri-dish or other suitable mediums.

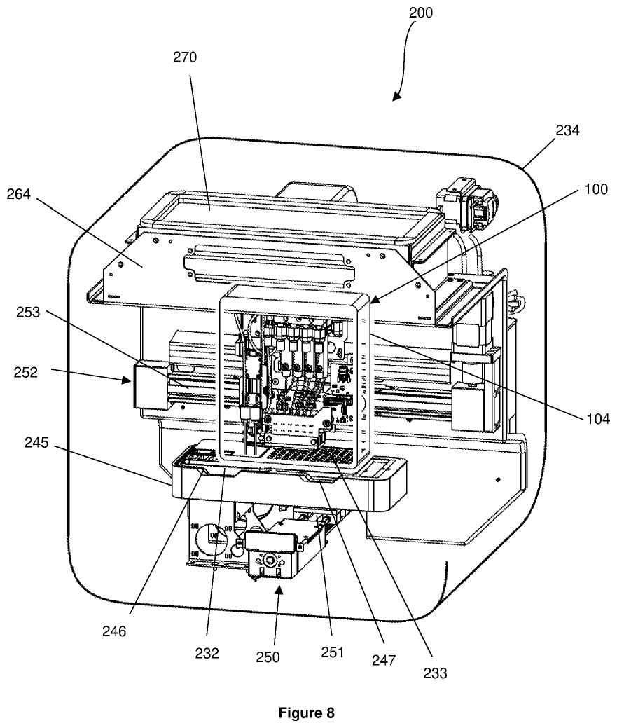

Referring to FIGS. 8 and 10, the housing 234 has a holder 245 having a receptacle 246 and a print stage 247. A cartridge 232 is removably received in the receptacle 246 and the substrate 233 is removably supported on the print stage 247. The holder 245 has a reader (not shown) that is electrically connected to the control system 272 (discussed below). When a cartridge 232 is received in the receptacle 246, the reader is configured to read the identifier 244 of the cartridge 232 to obtain information about the cartridge 232 and pass this information onto the control system 272.

The reader may be a read/write RFID or NFC reader that is capable of reading and rewriting information on a respective RFID or NFC tag or label. In the case where the identifier 244 is a read-only RFID of NFC tag or label, the read/write RFID of NFC reader can only obtain information from the respective RFID or NFC tag or label. In the case where the identifier 244 is a rewritable RFID of NFC tag or label, the read/write RFID of NFC reader is able to obtain information from, and rewrite information on, the respective rewritable RFID of NFC tag or label.

Referring to FIGS. 9 and 10, the base 236 of the cartridge 232 has a chamfer 248 and the corner 249 of the receptacle 246 has a shape that complements the chamfer 248. It will be appreciated that the chamfer 248 and the corner 249 cooperate such that the cartridge 232 can only be inserted into the receptacle 246 in a certain orientation, which prevents the sealed containers 238, the unsealed containers 239, the cleaning container 240, and the waste slot 241 being incorrectly oriented in the receptacle 246.

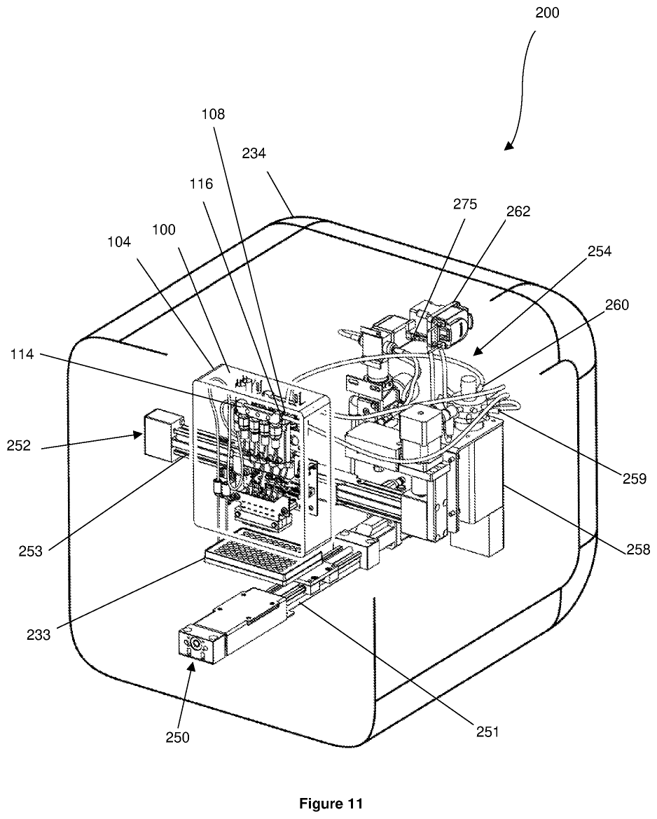

Referring to FIGS. 8 and 11, the housing 234 has a first positioning unit 250 coupled to the holder 245. The first positioning unit 250 has a track 251 and is configured to move/position the holder 245 anywhere along the length of the track 251. It will therefore be appreciated that the first positioning unit 250 is capable of moving/positioning the cartridge 232 and the substrate 233 anywhere along the length of the track 251.

The housing 234 also has a second positioning unit 252 coupled to the printhead housing 104. The second positioning unit 252 has a track 253 and is configured to move/position the printhead assembly 100 anywhere along the length of the track 253. The track 253 of the second positioning unit 252 extends substantially perpendicular to the track 251 of the first positioning unit 250. The first positioning unit 250 and the second positioning unit 252 together allow the printhead assembly 100 to be positioned/moved over the cartridge 232 and/or the substrate 233.

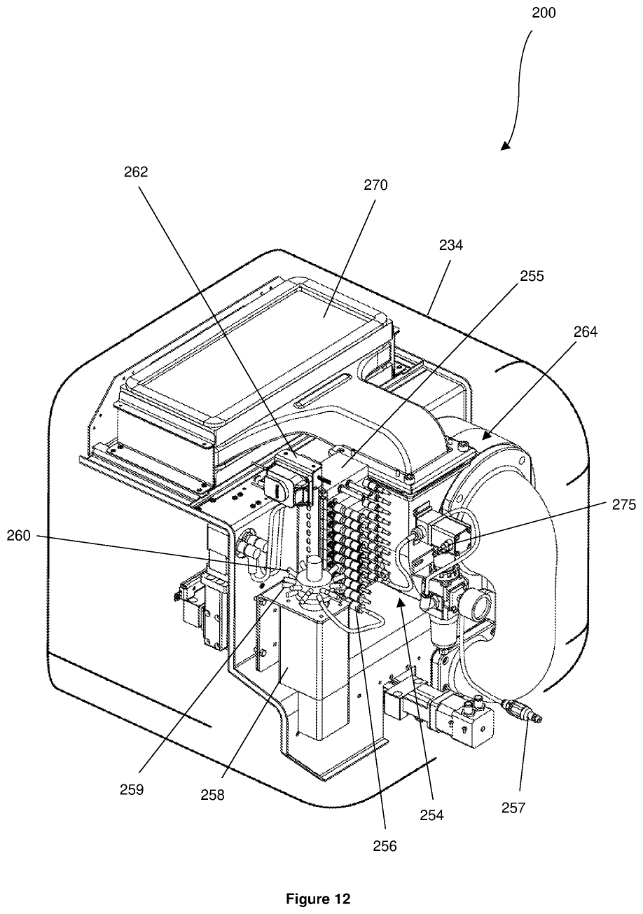

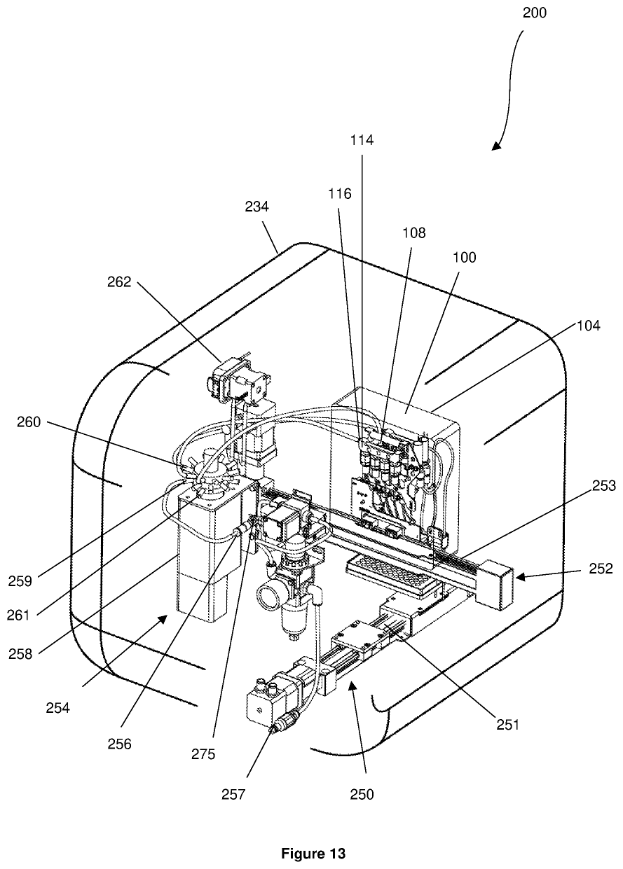

Referring to FIGS. 11 to 13, a pressure regulating system 254 is disposed in the housing 234. The pressure regulating system 254 has a regulator manifold 255 having a plurality of pressure regulators 256. The pressure regulating system 254 also has a connector 257 projecting from the housing 234. The connector 257 is in fluid communication with the regulator manifold 255 and is configured to be coupled in fluid communication to a source of pressurized gas. The source of pressurized gas may be, for example, an air compressor or a pump.

A selector valve 258 is disposed in the housing 234 and has a plurality of input connections 259, a plurality of output connections 260, and a plurality of channels 261 that can be selected by the selector valve 258.

Each pressure regulator 256 is coupled in fluid communication to one of the input connections 259 of the selector valve 258. The cap 108 of each reservoir 106 is coupled in fluid communication to one of the output connections 260 of the selector valve 258. The selector valve 258 therefore couples the interior of each reservoir 106 in fluid communication to one of the pressure regulators 256 of the pressure regulating system 254. Accordingly, the interior of each reservoir 106 is capable of being pressurized by the source of pressurized gas coupled to the connector 257. Each pressure regulator 256 regulates the pressure in the respective reservoir 106 and is capable of increasing and decreasing the pressure in the respective reservoir 106.

The manifold outlet 116 of each priming manifold 114 is coupled in fluid communication to one of the output connections 260 of the selector valve 258, such that each manifold outlet 116 is in fluid communication with one of the pressure regulators 256. Each subsystem 111 of the sample loading system 102 is therefore in fluid communication with the pressure regulating system 254. Accordingly, each subsystem 111 of the sample loading system 102 is capable of receiving pressurised gas from the source of pressurised gas coupled to the connector 257.

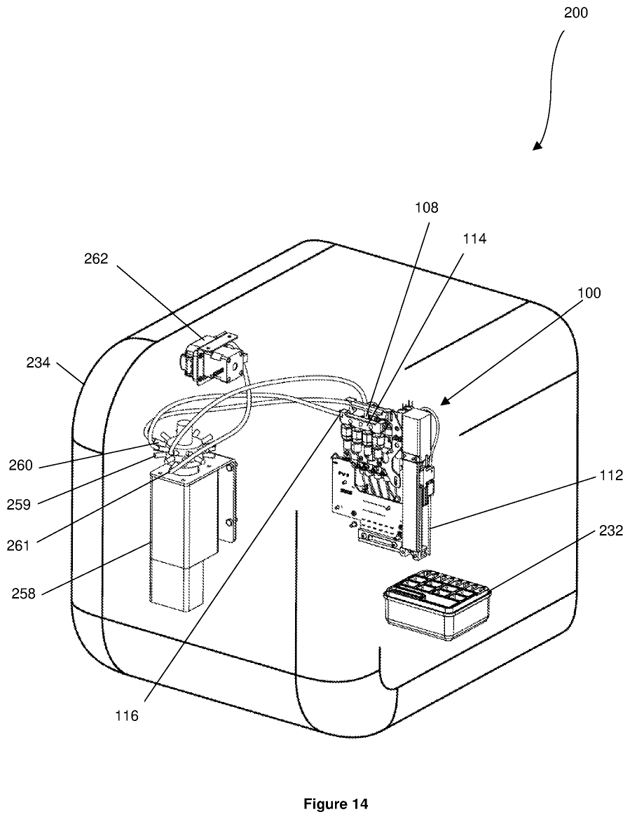

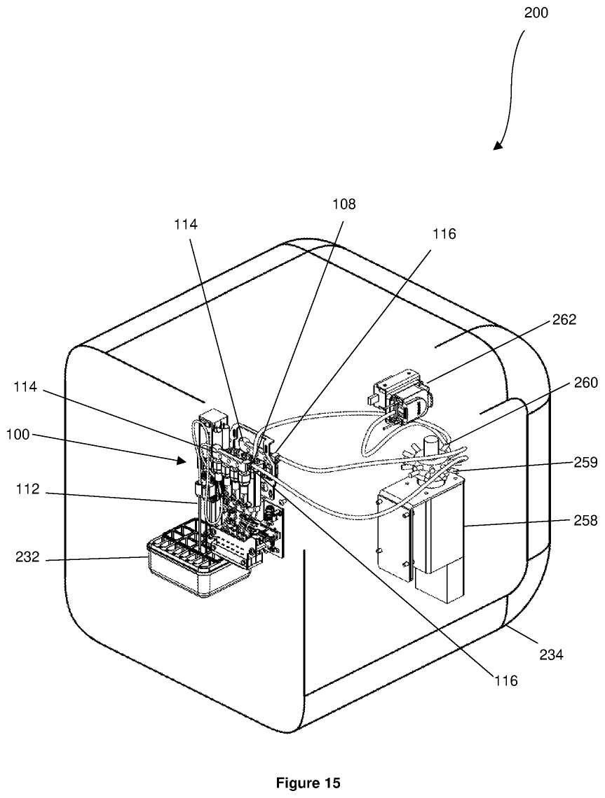

Referring to FIGS. 14 and 15, disposed in the housing 234 is a printer pump 262 coupled in fluid communication to one of the channels 261 of the selector valve 258. The selector valve 258 is capable of selectively coupling the channel 261 that is coupled to the printer pump 262 in fluid communication with either manifold outlet 116 of both priming manifolds 114. In this scenario, it will be appreciated that the printer pump 262 is in fluid communication with the sample loading system 102 via the respective manifold outlet 116. When the priming manifold channels 261 that are coupled to the printer pump 262 is not selected, the printer pump 262 is not in fluid communication with either manifold outlet 116 of both the priming manifolds 114 and the manifold outlets 116 are in fluid communication with the pressure regulating system 254.

The selector valve 258 is also capable of selectively coupling the cap 108 of each reservoir 106 in fluid communication with the printer pump 262. When the printer pump 262 is in fluid communication with the cap 108 of a reservoir 106, the printer pump 262 is configured to apply a negative or a positive pressure to the interior of the reservoir 106.

Referring to FIG. 6, the housing 234 has an access door 263 having an open position and a closed position. In the open position, the access door 263 permits access to the print area 276 within the housing 234. In the closed configuration, the access door restricts/prevents access to the print area 276 within the housing 234.

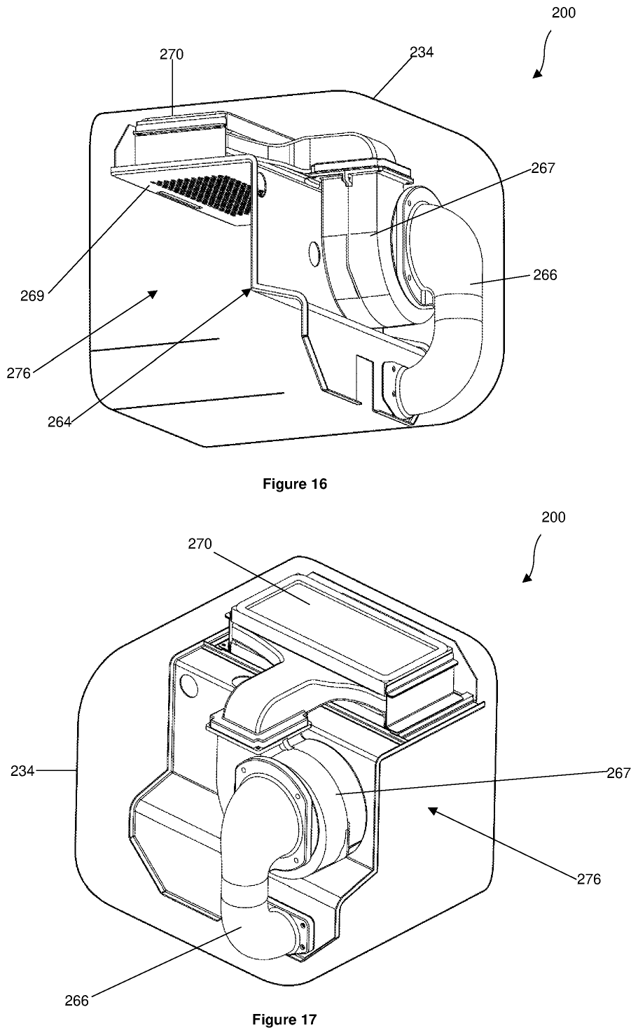

Referring to FIGS. 16 to 18, a laminar air flow system 264 is disposed in the housing 234. The laminar air flow system 264 has a first flow path 265 extending underneath the holder 245, a second flow path 266 isolated from and extending behind the print area 276, a blower 267 to induce an airflow within the housing 234, a grate 268 located below the holder 245 (see FIG. 6), a recycle High Efficiency Particulate Arresting (HEPA) filter 269 in fluid communication with the interior of the housing 234, and an exhaust HEPA filter 270 in fluid communication with an ambient environment.

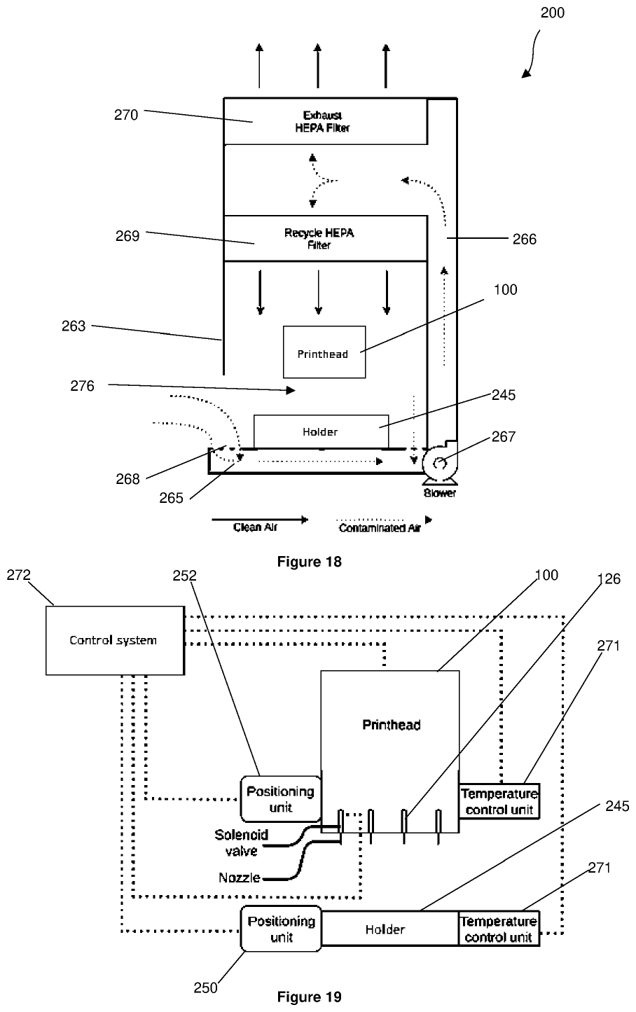

Referring to FIG. 18, the blower 267 is in fluid communication with the first flow path 265 and the second flow path 266. The blower 267 is configured to induce an air flow underneath the holder 245 by drawing potentially contaminated air into the first flow path 265 through the grate 268. The blower 267 is configured to force an airflow through the second flow path 266 by pumping the contaminated air into the second flow path 266. The flow rate of the air flowing through the first flow path 265 and the second flow path 266 can be increased and decreased by increasing or decreasing the revolutions per minute (rpm) of the blower 267, respectively.

As best seen in FIG. 18, external air drawn into the housing 234 is drawn into the first flow path 265 and flows underneath the holder 245. This reduces the amount of external air and, therefore, airborne contaminants flowing over the substrate 233 that could potentially contaminate the substrate 233 and any 3D cell construct printed on the substrate 233.

Air flowing through the second flow path 266 is either directed back into the print area 276 of the housing 234 through the recycle HEPA filter 269 or out of the housing 234 through the exhaust HEPA filter 270. The recycle HEPA filter 269 and the exhaust HEPA filter 270 remove a significant amount of particulates from the air flowing through them. Accordingly, air flowing back into the print area 276 of the housing 234 through the recycle HEPA filter 269 is sterile and contains a very low concentration of particulates. The air flowing from the recycle HEPA filter 269 into the print area 276 of the housing 234 is a unidirectional downward airflow through the print area 276 of the housing 234. This airflow provides a laminar airflow through the print area 276 of the housing 234, which may reduce the risk of the substrate 233 and any 3D cell construct printed on the substrate 233 being contaminated. It is envisaged that the unidirectional airflow through the print area 276 the housing 234 has a velocity of about 0.45 m/s.

Referring to FIG. 19, the bioprinter 200 has two temperature control units 271 that are disposed in the housing 234. One of the temperature control units 271 is disposed proximate the printhead assembly 100 and the other temperature control unit 271 is disposed proximate the holder 245.

The temperature control units 271 are capable of regulating the temperature within the housing 234 of the bioprinter 200 by providing heating or cooling, based on the conditions needed for sustained viability and/or optimal growth conditions for the cells to be printed by the bioprinter 200. For example, the temperature control units 271 can maintain the temperature in the housing 234 within a temperature range of about 36 to 38 degrees Celsius to assist cell proliferation of the printed cells.

The temperature control unit 271 disposed proximate the printhead 100 is also capable of maintaining the temperature of fluids contained in the reservoirs 106 within a predetermined temperature range. For example, this may be done to keep fluids contained in the reservoirs 106 above a predetermined temperature to promote cell proliferation in the printed cells and to keep the viscosity of fluids contained in the reservoirs 106 within a suitable range for printing.

The temperature control unit 271 disposed proximate the holder 245 is capable of maintaining the temperature of a substrate 233 disposed on the print stage 247 of the holder 245 within a predetermined range to promote cell proliferation in the printed cells for example.

It will be appreciated that the temperature control units 271 may cooperate to maintain the temperature within the housing 234 of the bioprinter 200 within a particular temperature range, or that they may operate independently to maintain the printhead 100 and substrate 233 within respective predetermined temperature ranges.

Still referring to FIG. 19, the bioprinter 200 is controlled by a control system 272 having custom software developed for printing 3D cell constructs. The control system 272 includes a non-transitory computer readable medium on which programs and algorithms for operating the bioprinter 200 are stored. It is envisaged that the non-transitory computer readable medium is located separately from the bioprinter 200 and is electrically connected to the bioprinter 200. It is also envisaged that the non-transitory computer readable medium may be provided with the bioprinter 200.

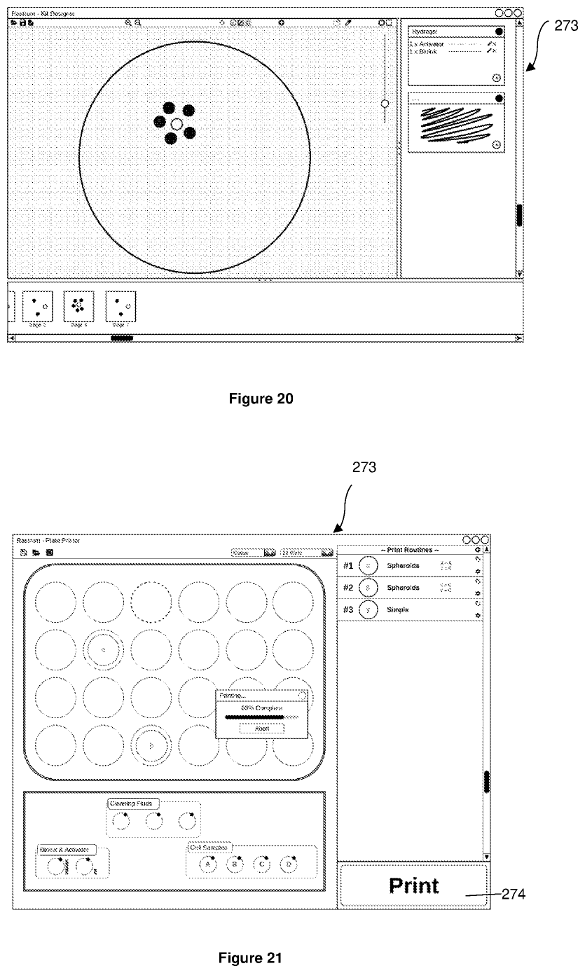

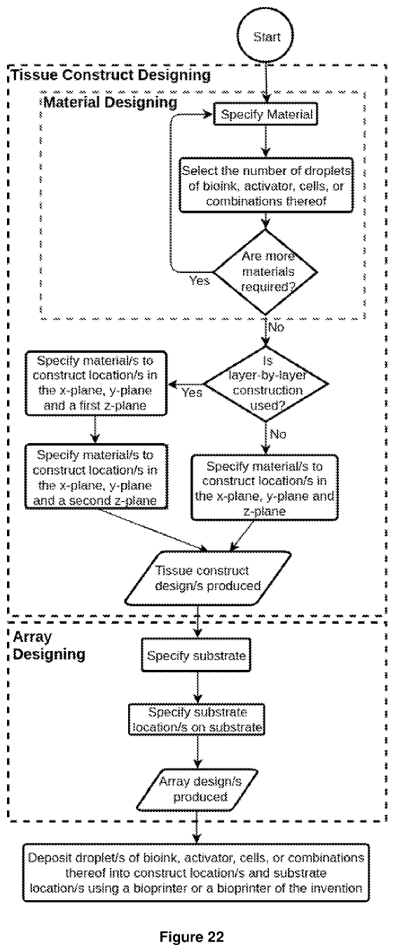

Referring to FIGS. 20 and 21, the control system 272 includes a graphical user interface (GUI) 273. Through the GUI 273, a user can select different printing routines and change parameters for printing particular 3D cell constructs. For example, the user can use the GUI 273 to change the spacing and the volume of the fluid droplets dispensed from the printhead assembly 100. The user can also manually control the spatial position of the fluid droplets dispensed from the printhead assembly 100 and create a custom pattern of fluid droplets to be dispensed from the printhead assembly 100 through the GUI 273. The control system 272 also includes operation instructions for cleaning, priming, and purging the first and second set of reservoirs 101, the sample loading system 102, and the dispensing system 103.

The GUI 273 allows a user to input instructions and information into the control system 272. For example, the user may input what fluids are in each of the sealed containers 238 and in which specific sealed containers 238 those fluids are located. The user may also input what fluids the user has added into each of the unsealed containers 239 and in which specific unsealed containers 239 those fluids are located. This allows the control system 272 to know where each fluid is located in the cartridge 232, such that the control system 272 can dispense the correct fluids from the printhead assembly 100 to fabricate the requisite 3D cell construct.

It will be appreciated that bioprinters print 3D cell constructs layer by layer. The intention behind layering of 3D cell constructs is to mimic how biologists use z-stack layering in a microscope. The GUI 273 provides the user with a method to design each layer of the 3D cell construct to be printed. For example, the GUI 273 provides a grid for the user to draw a pattern for each layer of the 3D cell construct to be printed.

As described above, the substrate 233 is a multi-well plate having a plurality of wells. Referring to FIG. 20, for example, the GUI 273 displays a visualization of the wells of the substrate 233 and predetermined 3D cell constructs that can be printed in each well of the substrate 233. Using the GUI 273, the user selects one well or an array of wells and a 3D cell construct to be printed in the well or the array of wells.

The GUI 273 allows a user to select where in/on the substrate 233 they would like to fabricate a 3D cell construct. The GUI 273 has a print preview button 274 that displays a visualization of where the cells of the 3D cell construct are going to be printed and what the 3D cell construct will look like. Once the user is satisfied with the visualization of the 3D cell construct on the GUI 273, the user can confirm that they would like to print the 3D cell construct through the GUI 273. The bioprinter 200 will then print the 3D cell construct on the substrate 233. The bioprinter will print 20 to 25 layers when fabricating the 3D cell construct, however, the user may increase or decrease the number of layers printed through the GUI 273.

The control system 272 is electrically connected to each sensor 117 and the electrical port 130 of the electronics assembly 129 in the printhead assembly 100. The control system 272 is also electrically connected to, and configured to control, both manifold valves 113, the actuator 122, each dispensing outlet 126, the first positioning unit 250, the second positioning unit 252, each pressure regulator 256, the selector valve 258, the printer pump 262, the blower 267, and the reader of the holder 245.

The electrical connector 131 of the electronics assembly 129 may be electrically connected to an electronics assembly (not shown) disposed in the housing 234 of the bioprinter 200 or to an electronics assembly (not shown) associated with the control system 272.

The bioprinter 200 is powered by a source of electric power removably coupled to the bioprinter 200. The source of electric power provides electric power to the electronics assembly 129, which distributes the electric power to the manifold valves 113, the sensors 117, the actuator 122, and each dispensing outlet 128. The source of electric power also provides electric power to the first positioning unit 250, the second positioning unit 252, the pressure regulating system 254, each pressure regulator 256, the selector valve 258, the printer pump 262, the blower 267, and the temperature control units 271. The source of electric power may be, for example, mains electricity.

Use and operation of the bioprinter 200 will now be described.

To print a particular 3D cell construct, a user selects a certain cartridge 232 that has the required bio-inks, activators, and other fluids needed to print the particular 3D cell construct contained in the sealed containers 238 of the cartridge 232. After the user has selected the appropriate cartridge 232, the user can add cell-inks, cell suspensions, cell culture media, and/or drugs in solution to any one of the unsealed containers 239 of the cartridge 232 by removing the lid 237 from base 236 of the cartridge 232. The user selects the fluids to add to each of the unsealed containers 239 depending on what the user is attempting to model with the particular 3D cell construct. After the user has added their chosen fluids to the unsealed containers 239, the user couples the lid 237 to the base 236 of the cartridge 232 to avoid contamination of the fluids contained in the unsealed containers 239.

Opening the access door 263 of the housing 234 allows the user to place the cartridge 232 into the receptacle 246 of the holder 245. When the access door 263 is in the open position, the user can also place the required substrate 233 onto the print stage 247 of the holder 245. After the user has placed the cartridge 232 into the receptacle 246 and the substrate 233 onto the print stage 247, the user removes the lid 237 of the cartridge 232 and closes the access door 263 of the housing 234.

When the access door 263 is in the open position, the control system 272 is configured to increase the rpm of the blower 267, which increases the flow rate of air through the housing 234. Increasing the rpm of the blower 267 also causes air flowing into the housing 234 through the open access door 263 to be drawn under the holder 245 through the grate 248 and into the first flow path 265. This reduces the amount of potentially contaminated air from entering into the housing 234 through the open access door 263 and flowing over and contaminating the substrate 233, the fluids contained in the unsealed containers 239, and any 3D cell construct printed on the substrate 233.

When the access door 263 is in the closed position, the control system 272 is configured to operate the blower 267 at a lower rpm compared to when the access door 263 is in the open position. Reducing the rpm of the blower 267 reduces the flow rate of air through the housing 234. Lower flow rates of air through the print area 276 of the housing 234 reduces the effect of dehydration on the substrate 233, the fluids contained in the cartridge 232, and any printed 3D cell construct printed on the substrate 233.

When the cartridge 232 is received in the receptacle 246, the control system 272 is configured to use the reader of the holder 245 to read the identifier 244 of the cartridge 232 to obtain information about the cartridge 232. From reading the identifier 244 of the cartridge 232, the control system 272 may be capable of determining what fluids are contained in each individual sealed container 238. The user uses the GUI 273 to input into the control system 272 what fluids have been added to each of the unsealed containers 239 so that the control system 272 knows where to located each of these fluids.

At this stage, the user can design the particular 3D cell construct to be printed using the GUI 273. Once the user is satisfied with the 3D cell construct they have designed, the user uses the GUI 273 to confirm that they would like the bioprinter 200 to commence printing the 3D cell construct.

The identifier 244 of the cartridge 232 may be configured to inform the control system 272 if the cartridge 232 is new, has been used, or has been spent. If the cartridge 232 is new, the control system 272 permits the user to print the required 3D cell construct. If the cartridge 232 is used, the control system 272 may be configured to display a prompt on the GUI 273 informing the user if there is enough fluid in the cartridge 232 to complete the required job. If there is enough fluid, the control system 272 permits the user to print the required 3D cell construct. If there is not enough fluid, the control system 272 may be configured to inform the user to replace the cartridge 232. If the cartridge 232 is spent, the control system 272 displays this information on the GUI 273 and informs the user to replace the cartridge 232.

Once printing of the 3D cell construct has been confirmed, the control system 272 pressurizes each reservoir 106 via the caps 108 using the respective pressure regulators 256 of the pressure regulating system 254. Pressurizing each reservoir 106 also pressurizes the respective priming fluid line 120, which forces the check valves 121 of each priming fluid line 120 into the closed position, which prevents fluid flowing from the priming manifolds 114 into the respective priming fluid lines 120.

The description below relates to each subsystem 111 of the sample loading system 102. To prime a reservoir 106 with a particular fluid, the control system 272 moves the holder 245 and/or the printhead assembly 100 using the first positioning unit 250 and/or the second positioning unit 252, respectively, such that the opening 124 and the needle 112 of the subsystem 111 are positioned above the particular container in the cartridge 232 containing the fluid to be held by the reservoir 106. The control system 272 then operates the actuator 122 to advance the point 123 of the needle 112 out of the printhead housing 104 through the opening 124, such that the point 123 of the needle 112 is inserted into and is submerged in the fluid contained in the particular container of the cartridge 232. It will be appreciated that if the required fluid is contained in one of the sealed containers 238 or the waste container 240, the point 123 of the needle 112 will puncture the seal 242 when the point 123 of the needle is being inserted into the respective sealed container 238 or waste container 240.

At this stage, the control system 272 opens the manifold valve 113 and controls the selector valve 258 to select the channel 261 that is coupled to the printer pump 262 to place the printer pump 262 in fluid communication with the manifold outlet 116 of the priming manifold 114 of the subsystem 111. The control system 272 then operates the printer pump 262 to apply a negative pressure to the manifold outlet 116 of the priming manifold 114, which causes a fluid slug to be drawn through the needle 112, through the manifold valve 113, and into the priming manifold 114 through the manifold inlet 115. The control system 272 continues to apply a negative pressure to the manifold outlet 116 of the priming manifold 114 until the sensor 117 detects that the fluid slug has begun to flow out of the manifold outlet 116, at which point, the control system 272 stops operation of the printer pump 262 and closes the manifold valve 113.

The sensor 117 can be disposed at the manifold outlet 116 to detect when the fluid slugs begins to flow out of the manifold outlet 116. Alternatively, the sensor 117 may be disposed at the manifold inlet 115 to detect when the fluid slug begins to flow into the manifold 114 through the manifold inlet 115. If the sensor 117 is disposed at the manifold inlet 115, the control system 272 may be configured to calculate the volume of the fluid slug that has flowed into the manifold 114 using the sensor 117. The control system 272 may then be configured to estimate when the fluid slug may begin to flow out of the manifold outlet 116 based on the volume of the manifold 114 and the volume of the fluid slug. It is also envisaged that a combination of a sensor 117 disposed at the manifold inlet 115 and a sensor 117 disposed at the manifold outlet 116 may be used.

The control system 272 subsequently controls the respective pressure regulator 256 to depressurize the reservoir 106 that is to be primed with the fluid slug and operates the printer pump 262 to apply a positive pressure to the manifold outlet 116 of the priming manifold 114. After the reservoir 106 has been depressurized, the positive pressure applied to the manifold outlet 116 of the priming manifold 114 by the printer pump 262 causes the check valve 121 of the respective priming fluid line 120 to move to the open position, whereby the fluid slug flows out of the priming manifold 114 through the respective priming fluid line 120 and into the depressurized reservoir 106. It will be appreciated that the positive pressure applied to the manifold outlet 116 of the priming manifold 114 by the printer pump 262 causes the fluid that has flowed out of the manifold outlet 116 to flow back into the priming manifold 114 and into the depressurized reservoir 106. The fluid slug in the depressurized reservoir 106 will flow into, and through, the respective dispensing fluid line 125 until it is stopped by the normally closed dispensing outlet 126 of the dispensing fluid line 125. At this stage, the depressurized reservoir 106 has been primed with the fluid slug and the control system 272 stops operation of the printer pump 262.

After the depressurized reservoir 106 has been primed, the control system 272 controls the respective pressure regulator 256 to increase the pressure in the depressurized reservoir 106, which moves the respective check valve 121 to the closed position to prevent fluid flowing from the priming manifold 114 into the reservoir 106.

As discussed above, the predetermined volume of each reservoir 106 may be sized to reduce, or possibly prevent, the fluid slug that has been pumped into the respective reservoir 106 flowing back up the respective priming fluid line 120.

After a reservoir 106 has been primed, the control system 272 opens the manifold valve 113 and operates the printer pump 262 or the respective pressure regulator 256 to apply a positive pressure to the priming manifold 114 and the needle 112 via the manifold outlet 116 to purge any fluid that remains in the subsystem 111 out through the needle 112. Any fluid remaining in the subsystem 111 can be purged back into the same container the fluid was initially drawn from or into the waste volume of the cartridge 232. If the fluid is to be purged into the waste volume, the control system 272 uses the first positioning unit 250 and/or the second positioning unit 252 to position the opening 124 and the needle 112 of the subsystem 111 above the waste slot 241 of the cartridge 232 before purging the subsystem 111. The control system 272 may be configured to operate the actuator 122 to insert the point 123 of the needle 112 into the waste slot 241 before purging the subsystem 111 to prevent/limit any purged fluids contaminating the substrate 233 or any of the fluids contained in the unsealed containers 239. After purging fluids from the subsystem 111 into the waste volume, the control system 272 operates the actuator 122 to retract the point 123 of the needle 112 back into the printhead housing 104 of the printhead assembly 100.

After the subsystem 111 has been purged of any fluids, the control system 272 may clean the subsystem 111 before priming another reservoir 106. To clean the subsystem 111, the control system 272 positions the printhead assembly 100 such that the needle 112 is located above the cleaning container 240 and operates the actuator 122 to advance the point 123 of the needle 112 until it punctures the seal 242 and is submerged in the cleaning fluid contained in the cleaning container 240. The control system 272 draws cleaning fluid through the needle 112 into the priming manifold 114 using a similar method to that described above. Subsequently, the control system 272 purges the cleaning fluid into the waste volume of the cartridge 232 using a similar method to that described above. The cleaning step described above may be repeated one or more times before priming another reservoir 106.

To prime further reservoirs 106, the control system 272 repeats the methods steps described above. Depending on the 3D cell construct to be printed, the control system 272 may prime each reservoir 106 or only a few of the reservoirs 106. The control system 272 may be configured to record the contents of each reservoir 106 so that the control system 272 knows which reservoirs 106 contain which fluids.

As each subsystem 111 is coupled to one set of reservoirs 101, it will be appreciated that the sample loading system 102 can simultaneously prime a reservoir 106 from the first set of reservoirs 101 and a reservoir 106 from the second set of reservoirs 101. The use of two subsystems 111 allows fluids that would react with each other and solidify to be handled by separate subsystems 111. For example, a bio-ink and an activator may react together and solidify to form a hydrogel. If the bio-ink and the activator are handled by the same subsystem 111, hydrogels may form in the subsystem 111, as the subsystem 111 may not be fully purged of a bio-ink before an activator is drawn through the subsystem 111. The formation of hydrogels in the subsystem 111 may result in blockages in the subsystem 111. Accordingly, having two, or more, subsystems 111 can reduce the possibility of this occurring.

So that reactive fluids are not handled by the same subsystem 111, reactive fluids are contained in adjacent containers in the cartridge 232, such that when the actuator 122 is operated to advance the needles 112, one needle 112 is inserted into a container containing one of the reactive fluids and the other needle 112 is inserted into an adjacent container containing the other reactive fluid.

Once the required reservoirs 106 have been primed with the fluids needed to fabricate the selected 3D cell construct, the control system 272 may then commence printing the 3D cell construct on/in the substrate 233. The control system 272 prints each layer of the 3D cell construct by dispensing certain fluids from the dispensing system 103 at specific times and locations through the print job. For example, the 3D cell construct may require particular materials to be fabricated by mixing/reacting multiple fluids held in different reservoirs 106. This may be achieved by dispensing a first fluid droplet from one reservoir 106 and dispensing a second fluid droplet from a second reservoir 106 onto the first fluid droplet. For example, a hydrogel can be formed by mixing a fluid droplet of bio-ink with a fluid droplet of an activator.

To dispense a particular fluid from the printhead assembly 100 at a specific location, the control system 272 positions the printhead assembly 100 using the first positioning unit 250 and/or the second positioning unit 252 such that the dispensing outlet 126 of the reservoir 106 holding the particular fluid is positioned above the specific location on the substrate 233. The control system 272 then moves the respective dispensing outlet 126 to the open configuration and the pressure within the reservoir 106 forces the fluid within the reservoir 106 to be dispensed from the dispensing outlet 126. Once the required volume of the particular fluid has been dispensed from the respective dispensing outlet 126, the control system 272 moves the dispensing outlet 126 back to the closed configuration to prevent further fluid being dispensed from the dispensing outlet 126.

It will be appreciated that dispensing fluid from a reservoir 106 will reduce the pressure in the reservoir 106. Accordingly, after fluid has been dispensed from a reservoir 106 and the respective dispensing outlet 126 is moved to the closed configuration, the control system 272 controls the respective pressure regulator 256 to re-pressurize the reservoir 106 to a predetermined pressure.

Increasing and decreasing the pressure within a reservoir 106 will increase and decrease the flow rate of fluid through the corresponding dispensing outlet 126, respectively. Increasing and decreasing the period of time the dispensing outlet 126 is in the open configuration will increase and decrease the volume of fluid dispensed from the dispensing outlet 126, respectively. Accordingly, it will be appreciated that the fluid droplet dispensed from the dispensing outlet 126 can be varied by varying the pressure within the respective reservoir 106 and varying the period of time the dispensing outlet 126 is in the open configuration. The control system 272 may be configured to control the volume of the fluid droplet dispensed from a particular reservoir 106 depending on the fluid contained in the reservoir 106 and the 3D cell construct to be printed. Alternatively, the user may control the volume of the fluid droplets dispensed from the printhead assembly 100 manually through the GUI 273 when designing the 3D cell construct.

The dispensing steps described above are repeated until all the fluid droplets required to fabricate the selected 3D cell construct have been dispensed. After the 3D cell construct has been fabricated, the control system 272 may be configured to update the information on the identifier 244 of the cartridge 232 to indicate that the cartridge 232 has been used and whether or not the cartridge may be used to print a further 3D cell construct. This updated information will be presented on the GUI 273 if the user attempts to use the cartridge 232 again to print a further 3D cell construct. At this stage, the user may remove the cartridge 232, the substrate 233, and any 3D cell constructed fabricated on the substrate 233, through the access door 263 of the housing 234.

After the 3D cell construct has been printed, the control system 272 is configured to purge any fluids remaining in the reservoirs 106. To purge a reservoir 106, the control system 272 positions the printhead assembly 100 using the first positioning unit 250 and/or the second positioning unit 252 such that the respective dispensing outlet 126 is located above the waste slot 241 of the cartridge 232. The control system 272 then purges all fluid remaining in the reservoir 106 into the waste volume of the cartridge 232 by dispensing the fluid using a similar method to that described above. This process is repeated until all the reservoirs 106 have been purged.

The control system 272 then primes each reservoir 106 with the cleaning fluid contained in the cleaning container 240 using a similar method to that described. The control system 272 then purges any cleaning fluid remaining in the subsystem 111 out through the needle 112 using a similar method to that described above. After the reservoirs 106 have been primed with cleaning fluid, the control system 272 dispenses all of the cleaning fluid from each reservoir 106 through the respective dispensing outlets 126 into the waste volume of the cartridge 232 using a similar method to that described above. The control system 272 may repeat the above cleaning process one or more times.

The control system 272 is capable of conducting and agitating/resuspension process to agitate/aerate fluids contained in the reservoirs 106. Where a fluid contained in a reservoir 106 is a suspension, the suspended particles in the suspension may settle, which may cause issues with the subsequently printed 3D cell construct or blockages in the bioprinter 200. The agitation/resuspension process causes any suspended particles that have settled to be resuspended.

To agitate/resuspend a fluid contained in a reservoir 106, the control system 272 controls the respective pressure regulator 256 to reduce the pressure in the reservoir 106. The control system 272 also closes a valve 275 in the pressure regulating system 254 to isolate the manifold outlets 116 from the source of pressurised gas connected to the connector 257. The control system 272 then controls the selector valve 258 to place the printer pump 262 in fluid communication with the cap 108 of the respective reservoir 106. The control system 272 then operates the printer pump 262 to apply a negative pressure to the reservoir 106 and opens the respective dispensing outlet 126. The negative pressure applied to the reservoir 106 causes the fluid in the respective dispensing fluid line 125 to flow back into the reservoir 106, and continued application of a negative pressure to the reservoir 106 causes air to be drawn into the reservoir 106 through the respective dispensing fluid line 125. Isolating the manifold outlets 116 from the source of pressurised gas connected to the connector 257 restricts/prevents air being drawn into the reservoir 106 through the respective priming fluid line 120 during the agitation/resuspension process, which would otherwise reduce the effective of this process.

The air drawn into the reservoir 106 bubbles through, and agitates, the fluid contained in the reservoir 106 before being drawn out of the reservoir 106 through the respective cap 108 by the printer pump 262. The control system 272 continues to apply a negative pressure to the reservoir 106 for a predetermined time that is sufficient to agitate/resuspend the fluid. After the fluid has been sufficiently agitated/resuspended, the control system 272 moves the respective dispensing outlet 126 to the closed configuration and stops operation of the printer pump 262. The control system 272 then opens the valve 275 and controls the selector valve 258 to place the cap 108 of the reservoir 106 back in fluid communication with its respective pressure regulator 256. Subsequently, the control system 272 controls this pressure regulator 256 to re-pressurize the reservoir 106 to a predetermined pressure.

It will be appreciated that the reservoirs 106 act as a degassing chamber. For example, when priming a reservoir 106 with a fluid slug, the configuration of the reservoir 106 will separate any air introduced into the reservoir 106 through the respective priming fluid line 120 from the fluid slug. This is because the denser fluid slug will flow to the lowest point in the reservoir 106 and displace any air that is introduced into the reservoir 106.

Due to the configuration of the sample loading system 102 and the first and second sets of reservoirs 101, it will be appreciated that each reservoir 106 can be refilled with a fluid without affecting any fluid already contained in the reservoir 106.

As the laminar air flow system 262 limits/prevents external contaminated air flowing over the substrate 233 and the cartridge 232, it will be appreciated that the bioprinter 200 does not need to be operated in a biosafety cabinet or a clean room facility. Accordingly, the cost associated with operating the bioprinter 200 can be reduced, as the bioprinter 200 can be operated in a standard room. The laminar air flow system 262 may also provide forced convective cooling to the printhead assembly 100 and its components, which may reduce, and possibly prevent, components in the printhead assembly 100 overheating and failing.

### Second Exemplary Embodiment of the Printhead Assembly

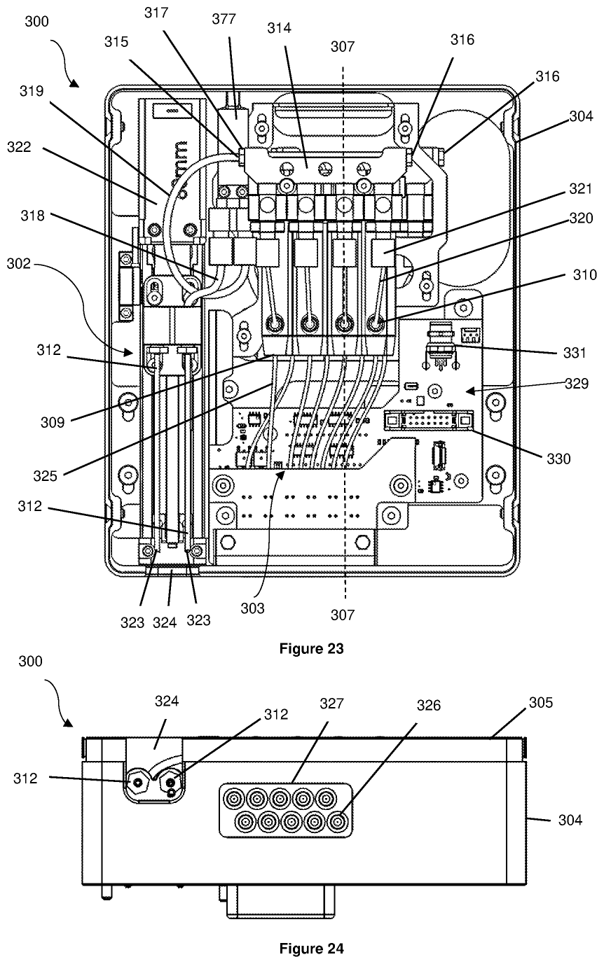

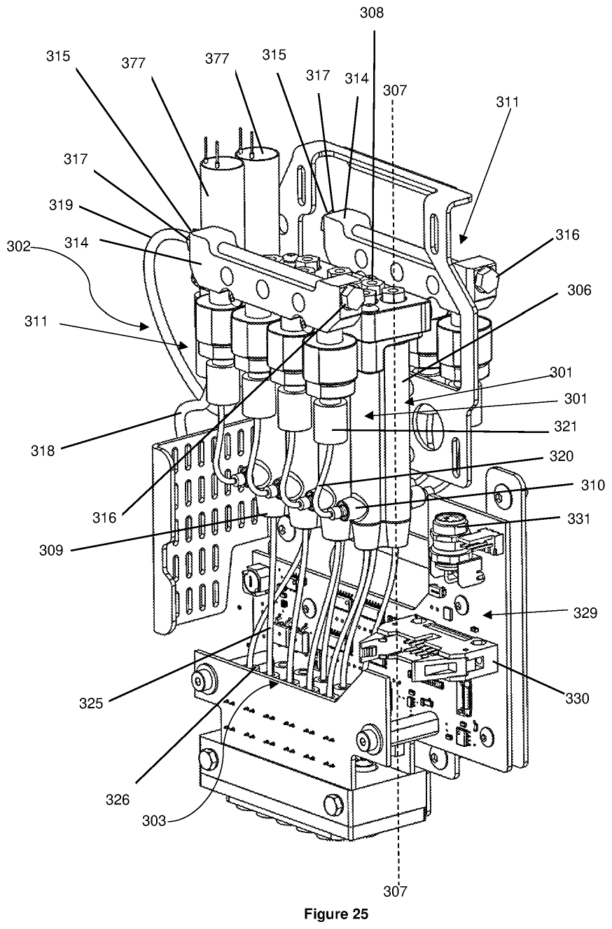

FIGS. 23 to 25 show a printhead assembly 300 according to a second embodiment of the present invention. The printhead assembly 300 is similar to the printhead assembly 100, except that the printhead assembly 300 has printhead pumps 377 instead of the manifold valves 113 of the printhead assembly 100 and that the manifold outlets 316 of the priming manifolds 314 are sealed in the printhead assembly 300.

Features of the printhead assembly 300 that are identical or equivalent to those of the printhead assembly 100 are provided with reference numerals that are equivalent to those of the printhead assembly 100 but incremented by 200. For features that are identical between the printhead assembly 100 and the printhead assembly 300, it will be appreciated that the above description of these features in relation to the printhead assembly 100 is also applicable to the corresponding identical/equivalent features found in the printhead assembly 300. Accordingly, the identical features between the printhead assembly 100 and the printhead assembly 300 will not again be described below in relation to the printhead assembly 300, as these features of the printhead assembly 300 have already been described above with respect to the printhead assembly 100.

For each subsystem 311 of the printhead assembly 300, the needle 312 is coupled in fluid communication to the printhead pump 377, which is coupled in fluid communication to the manifold inlet 315 of the priming manifold 314. The printhead pumps 377 may be positive displacement pumps such as, for example, peristaltic or diaphragm pumps, however, any other suitable pumps known in the art may be used.

The printhead assembly 300 can be used with the bioprinter 200. However, a bioprinter 200 using the printhead assembly 300 has a few structural differences compared to a bioprinter 200 using the printhead assembly 100. These structural differences are discussed below. For ease of reference, the bioprinter 200 using the printhead assembly 100 with be referred to below as “bioprinter 200” and the bioprinter 200 using the printhead assembly 300 will be referred to below as “bioprinter 200a”.

For the bioprinter 200a, the caps 308 of each reservoir 306 are coupled in fluid communication with one of the pressure regulators 256 of the pressure regulating system 254 via the selector valve 258. The printer pump 262 is also coupled in fluid communication with the selector valve 258. During normal operation of the bioprinter 200a, each of the caps 308 are in fluid communication with their respective pressure regulator 256. However, the control system 272 can control the selector valve 258 to place any one of the caps 308 in fluid communication with the printer pump 262. If one of the caps 308 is in fluid communication with the printer pump 262, that cap 308 is not in fluid communication with its respective pressure regulator and vice versa.

As the manifold outlets 316 of both priming manifolds 314 are sealed and the bioprinter 200a does not require the selector valve 258, the manifold outlets 316 of the priming manifolds 314 are not coupled in fluid communication to the pressure regulating system 254. Further, as the manifold outlets 316 of both manifolds 314 are sealed, the sensors 317 are disposed at the manifold inlets 315 of both manifolds 314 to detect fluid flowing into the priming manifolds 314 through the respective manifold inlets 315.

The operation and function of the bioprinter 200a is similar to that of the bioprinter 200, except for the way in which the reservoirs 306 are primed, the way in which the subsystems 311 are purged, and the agitation/resuspension process. The way in which the reservoirs 306 are primed and the way in which the subsystems 311 are purged is explained below. The description below relates to each subsystem 311 of the sample loading system 302.

To prime a reservoir 306 with a particular fluid, the control system 272 moves the holder 245 and/or the printhead assembly 300 using the first positioning unit 250 and/or the second positioning unit 252, respectively, such that the opening 324 and the needle 312 of the subsystem 311 are positioned above the particular container in the cartridge 232 containing the fluid to be held by the reservoir 306. The control system 272 then operates the actuator 322 to advance the point 323 of the needle 312 out of the printhead housing 304 through the opening 324, such that the point 323 of the needle 312 is inserted into and is submerged in the fluid contained in the particular container of the cartridge 232. It will be appreciated that if the required fluid is contained in one of the sealed containers 238, the point 323 of the needle 312 will puncture the seal 242 when the point 323 of the needle is being inserted into the respective sealed container 238.

At this stage, the control system 272 controls the respective pressure regulator 256 to depressurize the reservoir 306 that is to be primed with the desired fluid. The control system 272 subsequently controls the printhead pump 377 to draw a fluid slug through the needle 312 and printhead pump 377 and pump the fluid slug into the priming manifold 314 through the manifold inlet 315. As the reservoir 306 that is to be primed has been depressurized, the positive pressure applied to the manifold 314 by the printhead pump 377 causes the check valve 321 of the respective priming fluid line 320 to move to the open position, thereby causing the fluid slug to flow out of the priming manifold 314 through the respective priming fluid line 320 and into the depressurized reservoir 306. The fluid slug in the depressurized reservoir 306 will flow into, and through, the respective dispensing fluid line 325 until it is stopped by the normally closed dispensing outlet 326 of the dispensing fluid line 325. At this stage, the depressurized reservoir 306 has been primed with the fluid slug and the control system 272 stops operation of the printhead pump 377.

The control system 272 may be configured to utilize the sensor 317 to determine when the fluid begins to flow into the priming manifold 314 through the manifold inlet 315 and calculate the volume of fluid that has flowed into the manifold 314. The control system 272 may also be configured to utilize the sensor 317 to calculate the volume of fluid that has flowed into the depressurized reservoir 306.

After the depressurized reservoir 306 has been primed, the control system 272 controls the respective pressure regulator 256 to increase the pressure in the depressurized reservoir 306, which moves the respective check valve 321 to the closed position to prevent fluid flowing from the priming manifold 314 into the reservoir 306.

After a reservoir 306 has been primed, the control system 272 may be configured to clean the subsystem 311 and the respective manifold 314 before priming another reservoir 306. To clean the subsystem 311 and the respective manifold 314, the control system 272 effectively primes an empty reservoir 306 with cleaning fluid using a similar method to that described above. The control system 272 then dispenses the cleaning fluid from the respective reservoir 306 using a similar method to that described above with respect to the printhead assembly 100. This cleaning step may be repeated one or more times before priming another reservoir 306 with a fluid that is necessary to fabricate the selected 3D cell construct.

To prime further reservoirs 306, the control system 272 repeats the methods steps described above. It will be appreciated that, due to the printhead pumps 377 being disposed in the printhead assembly 300, priming of the reservoir 306 in the printhead assembly 300 may be faster compared to priming of the reservoirs 106 in the printhead assembly 100.

Similar to the bioprinter 200, the bioprinter 200a is also configured to perform an agitation/resuspension process. To agitate/resuspend a fluid contained in one of the reservoirs 306, the control system 272 controls the selector valve 258 to place the printer pump 262 in fluid communication with the cap 308 of the respective reservoir 306. The control system 272 then operates the printer pump 262 to apply a negative pressure to the reservoir 306 and opens the respective dispensing outlet 326. The negative pressure applied to the reservoir 306 causes the fluid in the respective dispensing fluid line 325 to flow back into the reservoir 306, and continued application of a negative pressure to the reservoir 306 causes air to be drawn into the reservoir 306 through the respective dispensing fluid line 325.

The air drawn into the reservoir 306 bubbles through, and agitates, the fluid contained in the reservoir 306 before being drawn out of the reservoir 306 through the respective cap 308 by the printer pump 262. The control system 272 continues to apply a negative pressure to the reservoir 306 for a predetermined time that is sufficient to agitate/resuspend the fluid in the reservoir 306. After the fluid has been sufficiently agitated/resuspended, the control system 272 moves the respective dispensing outlet 326 to the closed configuration and stops operation of the printer pump 262. The control system 272 then controls the selector valve 258 to place the cap 308 of the reservoir 306 back in fluid communication with its respective pressure regulator 256. Subsequently, the control system 272 controls this pressure regulator 256 to re-pressurise the reservoir 306 to a predetermined pressure.

It should be appreciated that the above description of the bioprinters 200, 200a using the printhead assemblies 100, 300 is to provide one example of how the printhead assemblies 100, 300 may be implemented and operated. It should also be appreciated that the printhead assemblies 100, 300 are not limited to use with the bioprinters 200,200a and may be used in other bioprinter types or examples.

Although the printhead assemblies 100, 300 has been described and illustrated as having two subsystems 111, 311 and a set of reservoirs 101, 301 coupled to each subsystem 111, 311, it will be appreciated that the printhead assemblies 100, 300 may have a sample loading system 102, 302 having a single subsystem 111, 311 coupled to a single set of reservoirs 101, 301 or more than two subsystems 111, 311, each of which being coupled to a respective set of reservoirs 101, 301.

It will also be appreciated that in its simplest form, the printhead assemblies 100, 300 have at least one reservoir 106, 306 in fluid communication with a sample loading system 102, 302 having a single subsystem 111, 311.

### Third Exemplary Embodiment of the Printhead Assembly

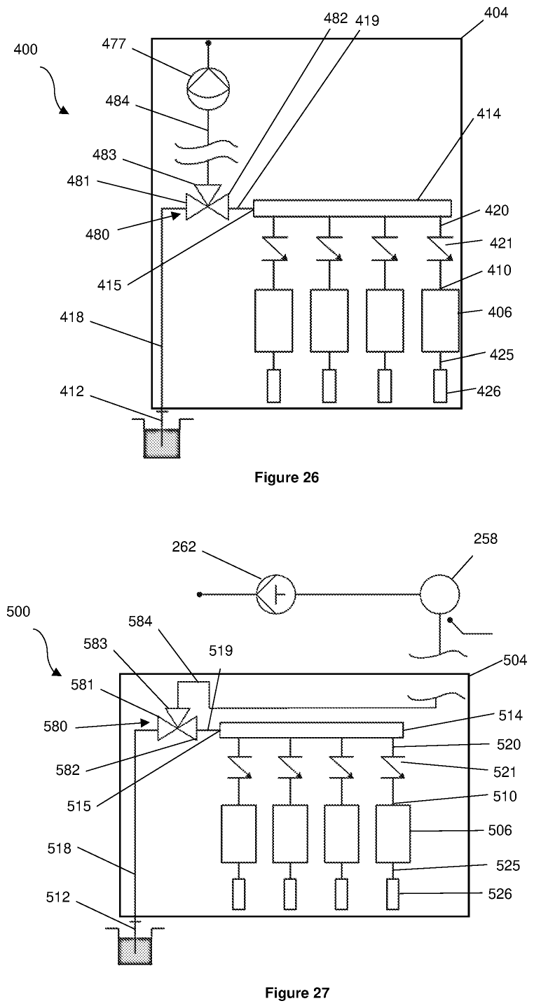

FIG. 26 shows a schematic of a printhead assembly 400 according to a third embodiment of the present invention. The printhead assembly 400 is similar to the printhead assembly 300, except that the printhead assembly 400 further comprises 3/2 valves 480.

Features of the printhead assembly 400 that are identical or equivalent to those of the printhead assembly 300 are provided with reference numerals that are equivalent to those of the printhead assembly 300 but incremented by 100. For features that are identical between the printhead assembly 400 and the printhead assembly 400, it will be appreciated that the above description of these features in relation to the printhead assembly 300 is also applicable to the corresponding identical/equivalent features found in the printhead assembly 400. Accordingly, the identical features between the printhead assembly 300 and the printhead assembly 400 will not again be described below in relation to the printhead assembly 400, as these features of the printhead assembly 400 have already been described above with respect to the printhead assembly 300.

Each subsystem 411 of the sample loading system 402 has a 3/2 valve 480. The 3/2 valve 480 of each subsystem 411 has a first port 481 coupled to the needle 412 by the fluid line 418, a second port 482 coupled to the manifold inlet 415 of the respective priming manifold 414 by the fluid line 419, and a third port 483 coupled to the printhead pump 477 by a fluid line 484.

The printhead assembly 400 can be used in the bioprinter 200a in the same way as described above. For ease of reference, a bioprinter 200a using a printhead assembly 400 will be referred to below as “bioprinter 200b”.

The operation of the bioprinter 200b is similar to that of the bioprinter 200a, except for the way in which the reservoirs 406 are primed and the way in which the subsystems 411 are purged. The description below describes these differences and relates to each subsystem 411 of the sample loading system 402.

To prime a reservoir 406, the control system 272 of the bioprinter 200b depressurises the reservoir 406 to be primed using the same method described above with respect to the bioprinter 200a. The control system 272 then controls the 3/2 valve 480 to place the needle 412 in fluid communication with the printhead pump 477. The control system 272 then controls the printhead pump 477 to draw a fluid slug up through the needle 412, through the fluid line 418, and into the fluid line 484. Subsequently, the control system 272 controls the 3/2 valve 480 to place the printhead pump 477 in fluid communication with the manifold inlet 415 of the respective priming manifold 414. The control system 272 then controls the printhead pump 477 to pump the fluid slug out of the fluid line 484, through the fluid line 419, and into the respective priming manifold 414 through the manifold inlet 415. As the reservoir 406 that is to be primed has been depressurized, the positive pressure applied to the manifold 414 by the printhead pump 477 causes the check valve 421 of the respective priming fluid line 420 to move to the open position, thereby causing the fluid slug to flow out of the priming manifold 414 through the respective priming fluid line 420 and into the depressurized reservoir 406. The fluid slug in the depressurized reservoir 406 will flow into, and through, the respective dispensing fluid line 425 until it is stopped by the normally closed dispensing outlet 426 of the dispensing fluid line 425. At this stage, the depressurized reservoir 406 has been primed with the fluid slug and the control system 272 stops operation of the printhead pump 477.

After the depressurized reservoir 406 has been primed, the control system 272 controls the respective pressure regulator 256 to increase the pressure in the depressurized reservoir 406, which moves the respective check valve 421 to the closed position to prevent fluid flowing from the priming manifold 414 into the reservoir 406.

After a reservoir 406 has been primed, the control system 272 may be configured to clean the subsystem 411 and the respective manifold 414 before priming another reservoir 406. To clean the subsystem 411 and the respective manifold 414, the control system 272 effectively primes an empty reservoir 406 with the cleaning fluid using a similar method to that described above. The control system 272 then dispenses the cleaning fluid from the respective reservoir 406 using a similar method to that described above with respect to the printhead assembly 100. This cleaning step may be repeated one or more times before priming another reservoir 406 with a fluid that is necessary to fabricate the selected 3D cell construct.

To prime further reservoirs 306, the control system 272 repeats the methods steps described above.

### Fourth Exemplary Embodiment of the Printhead Assembly

FIG. 27 shows a schematic of a printhead assembly 500 according to a fourth embodiment of the present invention. The printhead assembly 500 is similar to the printhead assembly 300, except that the printhead assembly 500 has 3/2 valves 580 instead of the printhead pumps 377 of the printhead assembly 300.

Features of the printhead assembly 500 that are identical or equivalent to those of the printhead assembly 300 are provided with reference numerals that are equivalent to those of the printhead assembly 300 but incremented by 200. For features that are identical between the printhead assembly 500 and the printhead assembly 500, it will be appreciated that the above description of these features in relation to the printhead assembly 300 is also applicable to the corresponding identical/equivalent features found in the printhead assembly 500. Accordingly, the identical features between the printhead assembly 300 and the printhead assembly 500 will not again be described below in relation to the printhead assembly 500, as these features of the printhead assembly 500 have already been described above with respect to the printhead assembly 300.

For the printhead assembly 500, each subsystem 511 of the sample loading system 502 has a 3/2 valve 580. For each subsystem 511, the 3/2 valve 580 has a first port 581 coupled to the needle 512 by the fluid line 518, a second port 582 coupled to the manifold inlet 515 of the respective priming manifold 514 by the fluid line 519, and a third port 583.

The printhead assembly 500 can be used in the bioprinter 200a in the same way as described above, except for one structural difference described below. For ease of reference, a bioprinter 200a using a printhead assembly 500 will be referred to below as “bioprinter 200c”.

For each subsystem 511 of the bioprinter 200c, the third port 583 of the 3/2 valve 580 is coupled to the selector valve 258 by a fluid line 584. For each subsystem 511, the control system 272 of the bioprinter 200c is configured to control the selector valve 258 to selectively place the third port 583 of the 3/2 valve 580 in fluid communication with the printer pump 262 of the bioprinter 200c.

The operation of the bioprinter 200c is similar to that of the bioprinter 200a, except for the way in which the reservoirs 506 are primed and the way in which the subsystems 511 are purged. The description below describes these differences and relates to each subsystem 511 of the sample loading system 502.

To prime a reservoir 506, the control system 272 of the bioprinter 200c depressurises the reservoir 506 to be primed using the same method described above with respect to the bioprinter 200a. The control system 272 then controls selector valve 258 to place the pump 262 in fluid communication with the third port 583 of the 3/2 valve 580. The control system 272 also controls the 3/2 valve 580 to place the third port 583 in fluid communication with the needle 512. The control system 272 then controls the printer pump 262 to draw a fluid slug up through the needle 512, through the fluid line 518, and into the fluid line 584. Subsequently, the control system 272 controls the 3/2 valve 580 to place the third port 583 in fluid communication with the manifold inlet 515 of the respective priming manifold 514. The control system 272 then controls the printer pump 262 to pump the fluid slug out of the fluid line 584, through the fluid line 519, and into the respective priming manifold 514 through the manifold inlet 515. As the reservoir 506 that is to be primed has been depressurized, the positive pressure applied to the manifold 514 by the printer pump 262 causes the check valve 521 of the respective priming fluid line 520 to move to the open position, thereby causing the fluid slug to flow out of the priming manifold 514 through the respective priming fluid line 520 and into the depressurized reservoir 506. The fluid slug in the depressurized reservoir 506 will flow into, and through, the respective dispensing fluid line 525 until it is stopped by the normally closed dispensing outlet 4526 of the dispensing fluid line 525. At this stage, the depressurized reservoir 506 has been primed with the fluid slug and the control system 272 stops operation of the printer pump 262.

After the depressurized reservoir 506 has been primed, the control system 272 controls the respective pressure regulator 256 to increase the pressure in the depressurized reservoir 506, which moves the respective check valve 521 to the closed position to prevent fluid flowing from the priming manifold 514 into the reservoir 506.

After a reservoir 506 has been primed, the control system 272 may be configured to clean the subsystem 511 and the respective manifold 514 before priming another reservoir 506. To clean the subsystem 511 and the respective manifold 514, the control system 272 effectively primes an empty reservoir 506 with the cleaning fluid using a similar method to that described above. The control system 272 then dispenses the cleaning fluid from the respective reservoir 506 using a similar method to that described above with respect to the printhead assembly 100. This cleaning step may be repeated one or more times before priming another reservoir 506 with a fluid that is necessary to fabricate the selected 3D cell construct.

To prime further reservoirs 506, the control system 272 repeats the methods steps described above.

### Cell Movement and Agitation/Resuspension Process

FIG. 26A shows a single unprimed (i.e., empty) reservoir 106, priming fluid line 120, and dispensing fluid line 125 of the printhead assembly 100. It has been found that the dispensing outlets 126, which are in the form of a nozzle, may have dead zones 178 under some cell printing situations. The dead zone 178 is a region within the dispensing outlet 126 where little to no fluid flow occurs.

FIG. 26B shows a single reservoir 106, priming fluid line 120, and dispensing fluid line 125 that have been primed with a cell suspension 10 having cells 12. As can be seen, the cell suspension 10 is homogenous. Referring to FIG. 26C, after a period of time, the cells 12 within the cell suspension 10 begin to settle and, as the fluid line 126 is substantially straight, the cells 12 settle in the dead zone 178 of the dispensing outlet 126.

FIG. 26D shows the agitation/resuspension process discussed above being applied to the reservoir 106 and dispensing fluid line 125. As can be seen, air 14 is bubbled up through the dispensing fluid line 125 and the reservoir 106. However, as there is little to no fluid flow in the dead zone 178, few, if any, of the cells 12 that have settled in the dead zone 178 are resuspended in the cell suspension 10, as can be seen in FIG. 26E. As there is little ability to resuspend the cells 12 that have settled in the dead zone 178, any 3D cell construct printed using the printhead assembly 100 may contain a lower concentration of cells 12 than expected, which may negatively impact the results obtained from the 3D cell construct.

FIG. 27A shows a single unprimed (i.e., empty) reservoir 106, priming fluid line 120 of the printhead assembly 100. In FIG. 27, the dispensing fluid lines 125 has been replaced with a dispensing fluid line 625. The dispensing fluid line 625 is similar to the dispensing fluid lines 125, expect that the dispensing fluid line 625 has a particulate trap 679. In one embodiment, the particulate trap 679 comprises a series of bends.

Features of the dispensing fluid line 625 that are identical or equivalent to those of the dispensing fluid line 125 are provided with reference numerals that are equivalent to those of the dispensing fluid line 125 but incremented by 500. For features that are identical between the dispensing fluid line 125 and the dispensing fluid line 625, it will be appreciated that the above description of these features in relation to the dispensing fluid line 125 is also applicable to the corresponding identical/equivalent features found in the dispensing fluid line 625. Accordingly, the identical features between the dispensing fluid line 125 and the dispensing fluid line 625 will not again be described below in relation to the dispensing fluid line 625, as these features of dispensing fluid line 625 have already been described above with respect to the dispensing fluid line 125.

FIG. 27B shows a single reservoir 106, priming fluid line 120, and the dispensing fluid line 625 that have been primed with a cell suspension 10 having cells 12. As can be seen, the cell suspension 10 is homogenous. Referring to FIG. 27C, after a period of time, the cells 12 within the cell suspension 10 begin to settle in the particulate trap 679. The particulate trap 679 therefore restricts/prevents the cells 12 from settling in the dead zone 678 of the dispensing outlet 626.

FIG. 27D shows the agitation/resuspension process discussed above being applied to the reservoir 106 and dispensing fluid line 625. As can be seen, air 14 is bubbled up through the dispensing fluid line 625 and the reservoir 106. As the majority of the cells 12 are trapped in the particulate trap 679, the air 14 being bubbled through the dispensing fluid line 625 transfers, and resuspends, the cells 12 back into the reservoir 106.

Referring to FIG. 26E, after the agitation/resuspension process has been completed, the cell suspension 10 within the reservoir 106 is homogenous. A homogenous cell suspension 10 within the reservoir 106 may allow a 3D cell construct to be printed having the desired concentration of cells 12, which may allow for more accurate results to be obtained from the printed 3D cell construct.

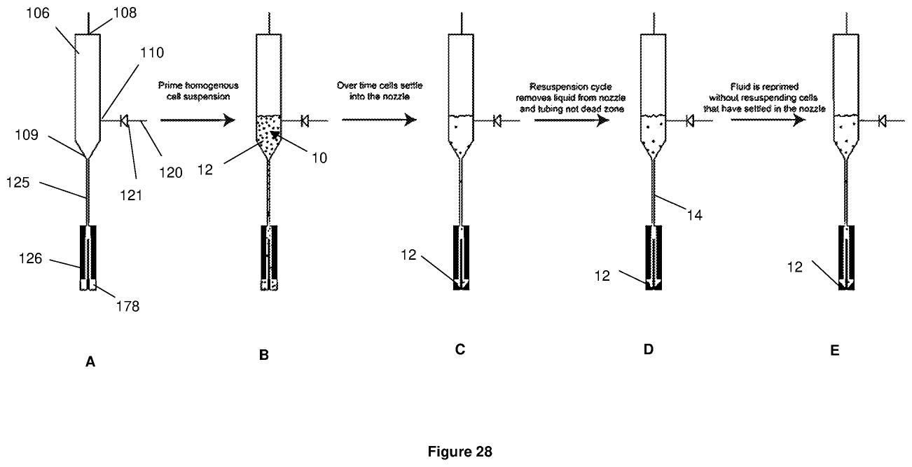

FIGS. 28A-C show dispensing fluid lines 625A-C having particulate traps 679 according to another embodiment. As can be seen in these figures, the particulate traps 679 are formed by creating one or more substantially vertical loops within the dispensing fluid line 625.

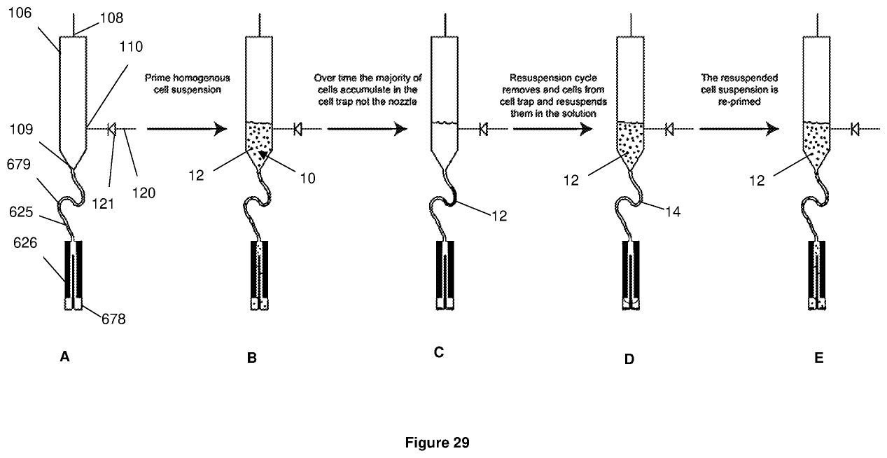

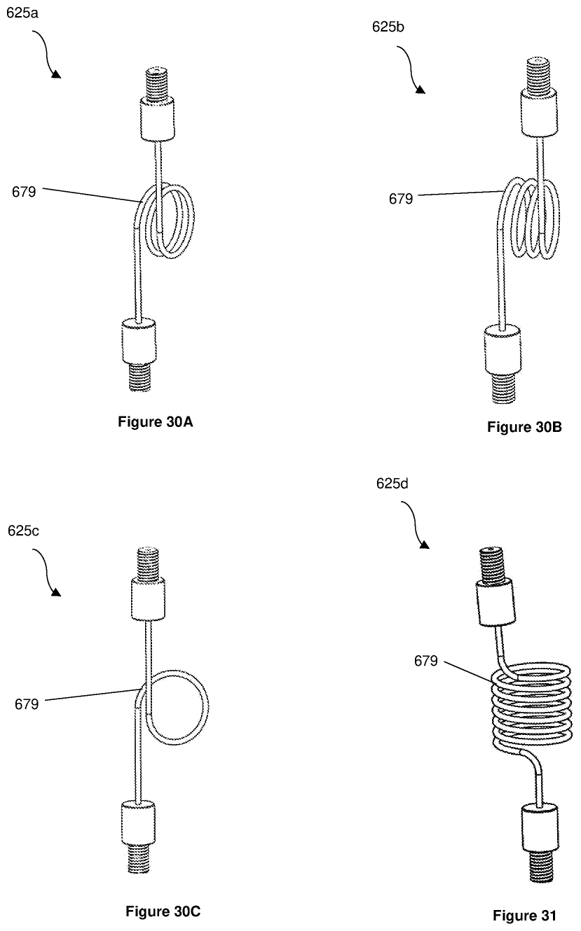

FIG. 29 shows a dispensing fluid line 625D having a particulate trap 679 according to another embodiment. As can be seen in this figure, the particulate trap 679 is formed by creating multiple horizontal loops in the dispensing fluid line 625. It is also envisaged that a single horizontal loop will suffice.

Although the dispensing fluid lines 625 have been described and illustrated with reference to the printhead assembly 100, it will be appreciated that the dispensing fluid lines 625 may also be used with the printhead assemblies 300, 400, 500 described above. Although the particulate trap 679 has been described as being used for trapping cells, it will be appreciated that the particulate trap 679 may be used for trapping other particulates suspended in a fluid suspension.

### Bio-Ink

In the present specification, bio-ink is defined as an aqueous solution of one or more types of macromolecule in which cells may be suspended or housed. Upon activation or crosslinking, it creates a hydrogel structure having its physical and chemical properties defined by chemical and physical composition of the bio-ink. Macromolecules are defined as an array of both synthetic and natural polymers, proteins and peptides. Macromolecules may be in their native state or chemically modified with amine or thiol-reactive functionalities.

Synthetic macromolecules may include:
Polysaccharides, such as polymers containing fructose, sucrose or glucose functionalities;
Non-ionic polymers, such as poly(ethylene glycol) (PEG), poly(hydroxyethyl methacrylate (PHEMA), poly(ε-caprolactone) (PCL), poly(vinyl alcohol) (PVA), poly(vinylpyrrolidone) (PVP), poly(NIPAAM) and poly(propylene fumarate) (PPF) and derivatives;
Polyelectrolytes—polymers that carry either positive or negative charge, amphoteric as well as zwitterionic polymer;
Polypeptides—a single linear chain of many amino acids (a minimum of 2 amino acids), held together by amide bonds; and
Nucleobase containing synthetic polymers—polymers with nucleobase (adenine, thymine, guanine or cytosine) repeating units.

Natural macromolecules may include:
Polysaccharides, such as alginate, chitosan, gellan gum, hyaluronic acid, agarose and glycosaminoglycan;
Proteins, such as gelatin, fibrin and collagen;
DNA and Oligonucleotides, such as single stranded DNA (ssDNA), double stranded DNA (dsDNA) DNAzymes and Aptamers; and
Basement membrane extracts.

Amine-reactive functionalities may include: aldehyde, epoxy, N-hydroxysuccinimide (NHS) and 2-vinyl-4,4-dimethylazlactone (VDM).

Thiol-reactive functionalities may include: alkenes, alkynes, azides, halogens and cyanates.

The bio-ink used and found suitable was alginate (at 2 w/v %) dissolved in calcium free DMEM supplemented with 10 v/v % FCS, L-glutamine and sodium pyruvate.

Bio-ink with dispersed SK-N-BE(2) neuroblastoma cells is referred to as bio-ink containing cells.

### Activator

In the present specification, an activator is an aqueous solution comprising of either small molecules or macromolecules which interact with the bio-ink to form a hydrogel structure. The composition of the activator can be altered to control the physical properties of the resulting hydrogel. The type of activator used is highly dependent on the macromolecules used as well as the intended crosslinking process.

Activators can be selected from:
Inorganic salts such as calcium carbonate, calcium chloride, sodium chloride, magnesium sulphate. sodium hydroxide and barium chloride;
Photoinitiators such as 2,2-dimethoxy-2-phenylacetophenone (DMPA) and Irgacure;
Polyelectrolytes—polymers that carry an opposite charge to the macromolecules in the bio-ink. It could be cationic, anionic, amphoteric and zwitterionic;
Polypeptides—a single linear chain of many amino acids (a minimum of 2 amino acids), held together by amide bonds;
DNA linker—macromolecules carrying nucleotides or DNA sequences which complement those present on the bio-ink's macromolecules; and
Natural or synthetic macromolecules carrying amine or thiol groups, either natively or through chemical modifications.

The activator used for the alginate bio-ink was calcium chloride at 4 w/v % dissolved in MilliQ water.

### Crosslinking or Gelation

This is the process whereby individual macromolecular chains are linked together by the activator to form a hydrogel. The crosslinking process can be classified to either chemical or physical crosslinking. Physical crosslinking or non-covalent crosslinking may include:
Ionic crosslinking—crosslinking via the interaction of the opposite charges present in the macromolecule and the activator. The activator may include charged oligomers, ionic salt and ionic molecule;
Hydrogen bonds—crosslinking via the electrostatic attractions of polar molecules. In this case, the macromolecule and the activator are carrying polar functionalities;
Temperature crosslinking—crosslinking via the rearrangement of the macromolecular chains as a response to change in temperature (heating or cooling); and
Hydrophobic interaction or van der Waals force.

Chemical or covalent crosslinking involves chemical reactions between the macromolecule and the activator. The type of reactions may include:
Photocrosslinking whereby the crosslinking reaction is promoted by UV or light irradiation;
Michael-type addition reaction between thiols and vinyl-carrying macromolecules in aqueous media;
Schiff base reaction between amino and aldehyde groups;
Diels-alder reaction;
Click chemistry;
Aminolysis reaction to active ester group; and
Enzyme crosslinking.

Examples of other bio-ink and activator combinations are set out in the Table below:

| Bio-Ink | Activator |
|---|---|
| Positively charged polyelectrolyte | Negatively charged polyelectrolyte |
| (e.g. poly(trimethylammonium) | (e.g. poly(sulfonate), |
| or poly(guanidium) | poly(carboxylic acid) |
| Fluorenylmethoxycarbonyl | Phosphate buffer solution |
| (Fmoc) polypeptide | Cell culture medium |
| Thiol-reactive macromolecules (e.g. | Photoinitiator and/or thiol- |
| PEG-diacrylate, hyaluronic acid | containing macromolecules |
| maleimide) | (e.g. bis-thiol-PEG) Thiol- |
| | containing polypeptides (e.g. |
| | bis-cysteine functionalised peptide) |
| Amine-reactive macromolecules | Amine-containing polypeptides |
| (e.g. PEG-co-Poly(benzaldehyde), | (e.g. bis-amine functionalised |
| aldehyde-alginate | peptide, gelatin, collagen) |
| Charged polysaccharides(e.g. | Inorganic salts (e.g. calcium |
| alginate and gellan gum) | chloride, barium chloride). |
| Macromolecules containing | Macromolecules containing the |
| nucleobase (e.g. Adenine) | corresponding nucleobase pair |
| | (e.g. Thymine) |

### Cell-Ink

In the present specification, cell-inks are an aqueous solution of one or more type of molecules or macromolecules in which cells are to be and remain evenly suspended throughout the 3D bio-printing process. The concentration of the cell-ink is optimised to prevent cells from settling but still maintains high cell viability.

Cell-link can be selected from:
Small molecules such as glycerol
Macromolecules such as Ficoll™, dextran, alginate, gellan gum, methylcellulose; and poly(vinylpyrrolidone) (PVP).

Ficoll™ is a neutral, highly branched, high-mass, hydrophilic polysaccharide which dissolves readily in aqueous solutions. Ficoll™ radii range from 2-7 nm and is prepared by reaction of the polysaccharide with epichlorohydrin. Ficoll™ is a registered trademark owned by GE Healthcare companies.

The cell-ink used was Ficoll™ 400 (at 10 w/v %) dissolved in PBS.

Cell-ink with dispersed SK—N-BE(2) neuroblastoma cells is referred to as cell-ink containing cells.

Gellan gum is a water-soluble anionic polysaccharide produced by the bacterium Sphingomonas elodea (formerly Pseudomonas elodea).

### Cell-Culture Solutions

In the present specification, cell-culture solutions are liquids that come into contact with the cultured cells and are suitable for various cell-related works. The preparation process includes careful analysis of the salt and pH balance, incorporation of only biocompatible molecules and sterilisation.

Some of the cell culture solutions include:
Cell culture medium such as Dulbecco's Modified Eagle Medium (DMEM), Minimum Essential Media (MEM), Iscove's Modified Dulbecco's Medium (IMDM), Media 199, Ham's F10, Ham's F12, McCoy's 5A and Roswell Park Memorial Institute (RPMI) medium;
Growth supplements such as foetal calf serum (FCS), epidermal growth factor (EGF), basic fibroblast growth factor (bFBF), fibroblast growth factor (FBF), endothelial cell growth factor (ECGF), insulin-like growth factor 1 (IGF-1) and platelet-derived growth factor (PDGF);
Biological buffers such as PBS, HEPES and CHES;
Chelating and stabilizing solutions; and
Sterilized MilliQ water.

### Cell-Culture Conditions

Cells and the 3D tissue culture models can be incubated, cultured and maintained using standard cell culture techniques. The 3D tissue culture models comprising the cells encapsulated in the hydrogel mold can be incubated under conditions to allow or maintain cell growth or spheroid formation. Incubation is typically carried out at about 37° C. with a CO2 level of 5% for at least 24 hours for most animal and human cell lines. It will be appreciated that incubation can be carried out at any suitable conditions, temperature and time duration that allows growth, maintenance or spheroid formation of the type of cell or cells in the hydrogel mold.

### Utility Solutions

Utility solutions are defined as the solutions which do not come into contact with the cells but are used to clean and sterilise all surfaces of the bioprinter 200 exposed to the cells. In other words, the utility solutions are cleaning fluids that may be contained in the cleaning container 240 of the cartridge 232. These solutions may include:
Ethanol at the correct concentration;
Sterile MilliQ water;
Cell culture medium;
Detergent; and
Hydrogen peroxide solution (2 w/v % maximum concentration).

### Preparation of Bio-Ink

Initially, bio-ink is prepared by mixing the right type and amount of macromolecules in the appropriate cell-culture solution. After achieving homogeneity, the blank bio-ink is sterilised via both UV irradiation and filtration (0.22 μm filter). The bio-ink is then kept at 4° C. until further usage.

### Preparation of Cells

Harvest cells by washing with PBS. Aspirate PBS. Add trypsin and incubate at 37° C. to dissociate cells from flask surface. Add tissue culture media to collect dissociated cells into a tube. Centrifuge cells, aspirate supernatant and resuspend pellet in fresh media. Perform cell count by mixing equal volumes of cell suspension and trypan blue stain. Perform calculation to determine the cell concentration. Desired numbers of cells then can be added to bio-ink, cell-ink or added to cell culture solutions.

### Preparation of Activators

The correct type and amount of molecules were dissolved in the appropriate cell-culture solution. The resulting solution was sterilised via UV irradiation and filtration prior to use.

### Preparation of Cell-Ink

The correct type and amount of molecules were dissolved in the appropriate cell-culture solution. After achieving homogeneity, the resulting solution was sterilised via UV irradiation and filtration prior to use. The cell-ink was then kept at room temperature until further use.

### Cell Harvesting

Cultured cells of interest at certain confluency are harvested by following the already established protocols. To make up the bio-ink or cell-ink containing cells, harvested cells are resuspended at the correct cell concentration to give 252 million cells/ml concentration in 200 μl of bio-ink or cell-ink. The resulting cell pellets are then redispersed in the correct volume of bio-ink or cell-ink. The bio-ink or cell-ink containing cells is then ready for use in the 3D bio-printer.

### Printing of Hydrogel Mold

The hydrogel mold can be printed using a drop-on-drop process whereby a droplet of bio-ink and a droplet of activator were deposited on top of each other to produce a hydrogel. This process can be repeated and used to form 3D hydrogel structures by building up layers of hydrogel.

### Cell Types

3D tissue culture models such as spheroids can be prepared from any suitable cell type including adherent cells such as mammalian liver cells, gastrointestinal cells, pancreatic cells, kidney cells, lung cells, tracheal cells, vascular cells, skeletal muscle cells, cardiac cells, skin cells, smooth muscle cells, connective tissue cells, corneal cells, genitourinary cells, breast cells, reproductive cells, endothelial cells, epithelial cells, fibroblast, neural cells, Schwann cells, adipose cells, bone cells, bone marrow cells, cartilage cells, pericytes, mesothelial cells, cells derived from endocrine tissue, stromal cells, stem cells, progenitor cells, lymph cells, blood cells, endoderm-derived cells, ectoderm-derived cells, mesoderm-derived cells, or combinations thereof.

Additional cell types may include other eukaryotic cells (e.g. chinese hamster ovary), bacteria (e.g. Helicobacter pylori), fungi (e.g. Penicillium chrysogenum) and yeast (e.g. Saccharomyces cerevisiae).

The cell line SK—N-BE(2) (neuroblastoma cells) has been used successfully in the process to produce 3D tissue culture models under a range of conditions. It will be appreciated that other cell lines would be expected to perform as required in 3D tissue models produced by the process developed. Other cell lines used include DAOY (human medulloblastoma cancer cells), H460 (human non-small lung cancer) and p53R127H (human pancreatic cancer cells). Other cell lines that may be suitable are listed on 088 and 089.

3D bio-printing technology was developed to produce high density 3D tissue culture models encapsulated in a hydrogel mold via drop-on-demand techniques. Specifically, a 3D printing technology was used to print biocompatible hydrogel molds using a bio-ink and activator that are constructed in a layer-by-layer manner to fabricate a variety of 3D structures. During the fabrication of the hydrogel molds, high cell density droplets can be included into the hydrogel mold.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

Although the invention has been described with reference to a preferred embodiment, it will be appreciated by persons skilled in the art that the invention may be embodied in many other forms. It will be appreciated by persons skilled in the art that numerous variations and/or modifications may be made to the technology as shown in the specific embodiments without departing from the spirit or scope of technology as broadly described. The present embodiments are, therefore, to be considered in all respects as illustrative and not restrictive.

1. A printhead assembly for a 3D bioprinter, the printhead assembly comprising:
a plurality of reservoirs;
a sample loading system having a manifold and a plurality of priming fluid lines, each priming fluid line coupling one reservoir in fluid communication to the manifold, wherein the manifold is configured to direct fluid into any one of the reservoirs and the sample loading system is configured to prime any one of the reservoirs with fluid; and
a dispensing system having a plurality of dispensing outlets, each dispensing outlet in fluid communication with one of the plurality of reservoirs and configured to dispense fluid from the respective reservoir.

2. The printhead assembly of claim 1, wherein the sample loading system is configured to draw a fluid from a container and prime any one of the plurality of reservoirs with the fluid.

3. The printhead assembly of claim 1, wherein:
each reservoir has a reservoir outlet and a reservoir inlet;
each dispensing outlet is in fluid communication with the reservoir outlet of one of the plurality of reservoirs; and
each priming fluid line is in fluid communication with the manifold and the reservoir inlet of one of the plurality of reservoirs.

4. The printhead assembly of claim 3, wherein each dispensing outlet is coupled in fluid communication to the reservoir outlet of one of the plurality of reservoirs by a dispensing fluid line.

5. The printhead assembly of claim 4, wherein each dispensing fluid line comprises a particulate trap configured to reduce particulates from settling in the respective dispensing outlet.

6. The printhead assembly of claim 5, wherein the particulate trap is one or more loops in the dispensing fluid line.

7. The printhead assembly of claim 1, wherein each priming fluid line comprises a valve having:
an open configuration that allows fluid to flow from the manifold into the respective reservoir; and
a closed configuration that prevents fluid flowing from the manifold into the respective reservoir.

8. The printhead assembly of claim 1, wherein the sample loading system comprises a pump coupled in fluid communication with an inlet of the manifold, the pump configured to draw fluid into the sample loading system and pump the fluid out of the sample loading system into any one of the reservoirs.

9. The printhead assembly of claim 8, wherein the sample loading system further comprises a needle in fluid communication with the inlet of the manifold, the needle configured to be inserted into a container to draw fluid from the container, and wherein the sample loading system further comprises an actuator configured to insert the needle into a container to draw fluid from the container and to withdraw the needle from the container.

10. (canceled)

11. The printhead assembly of claim 1, wherein each reservoir is configured to be coupled in fluid communication to a pressurized source of gas to pressurize each reservoir, and wherein each reservoir is configured to be coupled to a pressure regulator to regulate the pressure in the respective reservoir.

12. (canceled)

13. The printhead assembly of claim 1, wherein each dispensing outlet is a nozzle having:
an open configuration that allows fluid to be dispensed from the respective reservoir; and
a closed configuration that prevents fluid from being dispensed from the respective reservoir.

14. A 3D bioprinter for printing cells, the bioprinter comprising:
a printhead assembly according to claim 1;
a print stage for locating a substrate on which a 3D cell construct can be fabricated; and
a cartridge receptacle.

15. The bioprinter of claim 14, further comprising a housing in which the printhead assembly, the print stage, and the cartridge receptacle are disposed.

16. The bioprinter of claim 15, wherein the housing has an access door having an open position that permits access to an interior of the bioprinter and a closed position that prevents access to the interior of the bioprinter.

17. The bioprinter of claim 14, further comprising a pressure regulating system coupled in fluid communication to each reservoir to regulate the pressure in each reservoir, and the pressure regulating system configured to be coupled in fluid communication to a source of pressurized gas for pressurizing each reservoir.

18. The bioprinter of claim 15, further comprising an air flow system disposed in the housing, the air flow system configured to induce an air flow within the housing.

19. The bioprinter of claim 14, further comprising a holder in which the cartridge receptacle and the print stage are located.

20. The bioprinter of claim 19, further comprising a first positioning unit having a track, the first positioning unit coupled to the holder and configured to position the holder along the track of the first positioning unit.

21. The bioprinter of claim 14, further comprising a second positioning unit having a track, the second positioning unit coupled to the printhead assembly and configured to position the printhead assembly along the track of the second positioning unit.

22. A method of printing a three-dimensional (3D) cell construct by dispensing a plurality of fluid droplets from the dispensing system of a printhead according to claim 1.

23. (canceled)
